(19) United States
(12) Patent Application Publication
(10) Pub. No.: US 2021/0085801 A1
(43) Pub. Date: Mar. 25, 2021

(54) AMPHIPHILIC BLOCK COPOLYMERS, MICELLES, AND METHODS FOR TREATING OR PREVENTING HEART FAILURE

(71) Applicant: CARDIOL THERAPEUTICS INC., Oakville (CA)

(72) Inventors: Afsaneh LAVASANIFAR, Edmonton (CA); Anthony Ernest BOLTON, Oakville (CA)

(21) Appl. No.: 16/772,113

(22) PCT Filed: Dec. 10, 2018
(86) PCT No.: PCT/CA2018/051573

§ 371 Date: Jun. 11, 2020

Related U.S. Application Data
(60) Provisional application No. 62/597,740, filed on Dec. 12, 2017.

(51) Int. Cl. A61K 47/69; A61K 31/05; A61K 47/60; A61K 31/519; A61K 38/13; A61P 9/04

(52) U.S. Cl. CPC A61K 47/6907; A61K 31/05; A61P 9/04; A61K 31/519; A61K 38/13; A61K 47/60

(57) ABSTRACT

Micelle-forming amphiphilic block copolymers for use in targeting cardiac cells (e.g. fibrotic cells) of a subject suffering from heart failure, micelles containing the micelle-forming amphiphilic block copolymers together with a cardioactive agent, and related compositions and methods for treating or preventing heart failure, e.g. heart failure with preserved ejection fraction (HFpEF) also known as diastolic heart failure.

This application claims priority from, and the benefit of 35 U.S.C. 119(e) in respect of, U.S. provisional application 62/597,740 filed Dec. 12, 2017, the entire contents of which are incorporated herein by reference.

### FIELD

The present invention relates to amphiphilic block copolymers. In particular, the present invention relates to micelle-forming amphiphilic block copolymers as well as related compositions, methods, and uses.

### BACKGROUND

Polymeric micelles for carrying various agents such as small molecules, proteins, or DNA, have been described. For example, U.S. Pat. Nos. 8,309,515 and 9,139,553 relate to micelle-forming poly(ethylene oxide)-block-poly(ester) block copolymers having reactive groups on the polyester block therein. The biodegradability of these copolymers and their biocompatibilities with a large number of bioactive agents make them suitable as carriers for various bioactive agents. The bioactive agents, such as DNA, RNA, oligonucleotide, protein, peptide, drug and the like, can be coupled to the polyester block of the copolymer.

Several nanoparticles have been described as potentially useful for treating myocardial infarction. For example, International Patent Application Publication No. WO 2005/117561 relates to low density lipoprotein-like emulsions that bind to low density lipid receptors and their use in the diagnosis and treatment of a variety of diseases and disorders, including cardiovascular and ocular diseases. Other examples of such disclosures include Maranhao et al. (International Journal of Nanomedicine, 2017, 12:3767-3784); Suarez et al. (Biomater. Sci., 2015, 3(4):564-580); Geelen et al. (Contrast Media Mol. Imaging, 2013, 8:117-126); Lukyanov et al. (Journal of Controlled Release, 2004, 94:187-193); Harel-Adar et al. (Proc. Natl. Acad. Sci. U.S.A., 2011, 108(5):1827-1832); Dvir et al. (Nano Lett., 2011, 11(10):4411-4414); Lewis et al. (Proc. Natl. Acad. Sci. U.S.A., 2015, 112(9):2693-2698).

Ruiz-Esparza et al. (Eur. J. Heart Fail., 2016, 18(2):169-178) sought to investigate whether cardiovascular cells associate, internalize, and traffic a nanoplatform called mesoporous silicon vector (MSV), and determine its accumulation in cardiac tissue after intravenous administration in a murine model of heart failure. Results showed that fluorescence accumulated in failing myocardium, reaching intracellular regions of the cardiomyocytes. However, Guerrero-Beltran et al. (Am J Physiol Heart Circ Physiol., 2017, 312(4):H645-H661) found that although silicon dioxide (SiO₂) has emerged as a promising therapy vector for the heart, its potential toxicity and its mechanisms of damage remain poorly understood. This paper explored SiO₂-induced toxicity in cultured cardiomyocytes exposed to 7 nm or 670 nm SiO₂ particles and found that SiO₂ increases oxidative stress, which leads to mitochondrial dysfunction and low energy status.

A need exists for the development of a product, composition and/or method that provides the public with a useful alternative.

### SUMMARY

In accordance with an aspect, there is provided a micelle comprising a cardioactive agent, wherein the micelle is formed from an amphiphilic block copolymer; and the micelle, when administered systemically, preferentially and passively localizes in fibrotic tissue, and more preferentially localizes passively in association with cardiac fibroblasts.

In another aspect, the cardioactive agent is selected from the group consisting of anti-fibrotic agents, anti-inflammatory agents, statins, angiotensin receptor blockers, nitrates, beta-blockers, TLR4 antagonists, any blockers of HSP60 activity or inhibitors of production and/or transport of HSP 60, diuretics, inotropes, digoxin, vasodilators, angiotensin II converting enzyme (ACE) inhibitors, angiotensin II receptor blockers (ARB), calcium channel blockers, hydralazine, natriuretic peptides, cannabinoids, an anti-angiogenic agent, a vascular endothelial growth factor (VEGF) antagonist, a basic fibroblast growth factor (bFGF) antagonist, a bFGF receptor antagonist, a transforming growth factor-beta (TGF-β) antagonist, a TGF-β receptor antagonist, a steroidal anti-inflammatory agent, a non-pirfenidone TNF antagonist, tumor necrosis factor (TNF) antagonists, such as anti-TNF antibodies (e.g. REMICADE™ anti-TNF monoclonal antibody) and soluble TNF receptor (e.g. ENBREL™ TNF receptor-Ig immunoadhesin), and HUMIRA®, VEGF, bFGF, and TGF-beta, VEGF antagonists, VEGF receptor antagonists, bFGF antagonists, bFGF receptor antagonists, TGF-beta antagonists, and TGF-beta receptor antagonists, sildenafil, pirfenidone, rapamycin, methotrexate, amiodarone, cyclosporine, cyclosporine A, cyclosporine D, sacubitril/valsarten, soluble guanylate cyclase modulators, omecamtiv mecarbil, tacrolimus, valspodar, spironolactone, eplerenone, furosemide, dobutamine, milrinone, captopril, enalapril, lisinopril, benazepril, quinapril, fosinopril, ramipril, candesartan, irbesartan, olmesartan, losartan, valsartan, telmisartan, eprosartan, isosorbide mononitrate, isosorbide dinitrate, carvedilol, metoprolol, nesiritide, thalidomide, cannabidiol, derivatives thereof, and combinations thereof. In another aspect, the cardioactive agent is methotrexate or a derivative thereof, such as a lipophilic derivative thereof. In another aspect, the cardioactive agent is a cannabinoid, such as cannabidiol or a derivative thereof, such as a lipophilic derivative thereof. In another aspect, the cardioactive agent is a cyclosporin, such as cyclosporine A or cyclosporine D, or a derivative thereof such as Valspodar, or such as a lipophilic derivative thereof. In another aspect, the cardioactive agent is selected from the group consisting of sacubitril, valsarten, soluble guanylate cyclase modulators, omecamtiv mecarbil, tacrolimus, and combinations thereof. In another aspect, the cardioactive agent is hydrophilic or is a lipophilic derivative of a hydrophilic cardioactive agent, or wherein the cardioactive agent is lipophilic, and/or wherein the cardioactive agent is selected from cannabidiol, cyclosporine, derivatives thereof, and combinations thereof.

In another aspect, the amphiphilic block copolymer comprises a hydrophilic block selected from the group consisting of PEO (or PEG), PVP, derivatives thereof, and combinations thereof. In another aspect, the amphiphilic block copolymer comprises a hydrophobic block selected from the group consisting of a poly(ester), a poly(amino acid), a phospholipid, derivatives thereof, and combinations thereof. In another aspect, the amphiphilic block copolymer is selected from the group consisting of PEO-polycaprolactone, PEO-poly(valerolactone), PEO-poly(butyrolactone)s, PEO-polylactones, PEO-poly lactides, PEO-poly glycolides, PEO-polylactide-glycolide, PEO-poly(aspartic acid), PEO-poly(glutamic acid), 1,2-distearoyl-sn-glycero-3-phosphoethanolamine-N-[amino(polyethylene glycol) (PEG-DSPE), poly(ethylene oxide)-poly(caprolactone) (PEO-PCL), poly(ethylene oxide)-block-poly(α-benzyl carboxylate-ε-caprolactone) (PEO-PBCL), poly(ethylene oxide)-block-poly(α-carboxylate-ε-caprolactone) (PEO-PCCL), poly(ethylene oxide)-block-poly(α-cholestryl carboxylate-ε-caprolactone) (PEO-PChCL), derivatives thereof, and combinations thereof. In another example, wherein the amphiphilic block copolymer is poly(ethylene oxide)-poly(caprolactone) (PEO-PCL), poly(ethylene oxide)-block-poly(α-benzyl carboxylate-ε-caprolactone) (PEO-PBCL), poly(ethylene oxide)-block-poly(α-carboxylate-ε-caprolactone) (PEO-PCCL), derivatives thereof or combinations thereof. In another example, wherein the amphiphilic block copolymer is poly(ethylene oxide)-poly(caprolactone) (PEO-PCL), poly(ethylene oxide)-block-poly(α-benzyl carboxylate-ε-caprolactone) (PEO-PBCL), poly(ethylene oxide)-block-poly(α-carboxylate-ε-caprolactone) (PEO-PCCL), poly(ethylene oxide)-poly(caprolactone)-poly(α-propargyl carboxylate-ε-caprolactone) (PEO-PCL-PCC), poly(ethylene oxide)-block-poly(α-benzyl carboxylate-ε-caprolactone)-poly(α-propargyl carboxylate-ε-caprolactone) (PEO-PBCL-PCC), poly(ethylene oxide)-block-poly(α-carboxylate-ε-caprolactone)-poly(α-propargyl carboxylate-ε-caprolactone) (PEO-PCCL-PCC), derivatives thereof or combinations thereof. In another aspect, the amphiphilic block copolymer is PEO₁-PBCLₘ where n can be 10-300, 50-250, 75-200, 75-150, 100-150, or 100-125 and m can be 5-200, 5-150, 5-100, 10-100, 10-50, 10-30, 10-25 or 20-25. In another aspect, the amphiphilic block copolymer is PEOₙ-PCLₘ where n can be 10-300, 50-250, 75-200, 75-150, 100-150, or 100-125 and m can be 5-200, 5-150, 5-100, 10-100, 10-50, 10-30, 10-25 or 20-25. In another aspect, the amphiphilic block copolymer is PEOₙ-PCCLₘ where n can be 10-300, 50-250, 75-200, 75-150, 100-150, or 100-125 and m can be 5-200, 5-150, 5-100, 10-100, 10-50, 10-30, 10-25 or 20-25.

In another aspect, the amphiphilic block copolymer comprises a linker to accommodate a hydrophilic compound, such as by electrostatic complexation, hydrogen bonding, dipole-dipole bonding, or chemical conjugation. In another aspect, the linker comprises NH₂, SH, OH, or COOH.

In another aspect, the amphiphilic block copolymer comprises a compound of the formula I:

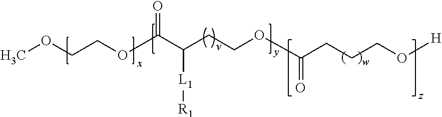

wherein
L₁ is a linker group selected from the group consisting of a single bond, —C(O)—O—, —C(O)—, —O—, —S—, —NH—, —NR²—, and —C(O)NR²;
R₁ is selected from the group consisting of H, OH, C₁₋₂₀ alkyl, C₃₋₂₀ cycloalkyl and aryl, said latter three groups may be optionally substituted and in which one or more of the carbons of the alkyl, cycloalkyl or aryl groups may optionally be replaced with O, S, N, NR² or N(R²)₂ or R₁ is a bioactive agent;
R² is H or C₁₋₆ alkyl;
v and w are independently of each other, an integer independently selected from 1 to 4;
x is an integer from 10 to 300;
y is an integer from 5 to 200;
z is an integer from 0 to 100;
wherein aryl is a mono- or bicyclic aromatic radical containing from 6 to 14 carbon atoms having a single ring or multiple condensed rings; and
wherein the optional substituents are selected from the group consisting of halo, OH, OC₁₋₆ alkyl, C₁₋₆ alkyl, C₂₋₆ alkenyl, C₂₋₆ alkenyloxy, NH₂, NH(C₁₋₆ alkyl), N(C₁₋₆ alkyl)(C₁₋₆ alkyl), CN, NO₂, C(O)C₁₋₆ alkyl, C(O)OC₁₋₆ alkyl, SO₂C₁₋₆ alkyl, SO₂NH₂, SO₂NHC₁₋₆ alkyl, phenyl and C₁₋₆ alkylenephenyl. In another aspect, L₁ is NH₂, SH, OH, or COOH. In another aspect, L₁ is —C(O)—O— or —C(O)—. In another aspect, R₁ is selected from the group consisting of optionally substituted C₁₋₆ alkyl, C₃₋₈ cycloalkyl, aryl in which one or more of the carbons of the alkyl, cycloalkyl or aryl groups may optionally be replaced with O, S or N, and a bioactive agent. In another aspect, the optional substituents are selected from the group consisting of halo, OH, OC₁₋₄alkoxy, C₁₋₄ alkyl, C₂₋₄ alkenyl, C₂₋₄ alkenyloxy, NH₂, NH(C₁₋₄ alkyl), N(C₁₋₄ alkyl)(C₁₋₄ alkyl), CN, NO₂, C(O)C₁₋₄ alkyl, C(O)OC₁₋₄ alkyl, SO₂C₁₋₄ alkyl, SO₂NH₂, SO₂NHC₁₋₄ alkyl, phenyl and C₁₋₄ alkylenephenyl. In another aspect, v and w are independently of each other, 2 or 3. In another aspect, v and w are equal. In another aspect, x is an integer from 50 to 200. In another aspect, x is an integer from 100 to 150. In another y is an integer from 5 to 100. In another aspect, y is an integer from 5 to 50. In another aspect, y is an integer from 10 to 20. In another aspect, z is an integer from 0 to 80, more suitably from 0 to 40.

In another aspect, the amphiphilic block copolymer comprises 2, 3, or more blocks. In another aspect, the block lengths are modified to effect desired qualities of the resultant micelle. In another aspect, the amphiphilic block copolymers are cross-linked. In another aspect, the amphiphilic block copolymers form a micelle around the cardioactive agent by one or more of chemical conjugation, electrostatic complexation, and physical encapsulation. In another aspect, the cardioactive agent is covalently bound or complexed to the amphiphilic block copolymer. In another aspect, the cardioactive agent is covalently bound or complexed to one or more monomers of the hydrophobic block of the amphiphilic block copolymer. In another aspect, the cardioactive agent is covalently bound or complexed within the hydrophobic block of the amphiphilic block copolymer. In another aspect, the cardioactive agent is covalently bound or complexed near or at the tail end of the hydrophobic block of the amphiphilic block copolymer. In another aspect, the cardioactive agent is complexed to the amphiphilic block copolymer by electrostatic complexation, hydrogen bonding, and/or dipole-dipole bonding.

In yet another aspect, the micelle has a size selected to localize to areas in which cardiac fibroblasts are present, such as a size of up to about 500 nm, or 250 nm and/or from about 10 nm, 25 nm, 50 nm, 75 nm, 100 nm, or 125 nm. For example, the size of the micelle can be about 125 nm, about 150 nm, about 175 nm, or about 200 nm.

In another aspect, each of the hydrophobic and hydrophilic blocks has a molecular weight of greater than about 2000 daltons, greater than about 3,000 daltons, or greater than about 5000 daltons, or, typically, from about 2000 to about 20,000 daltons.

In another aspect, the micelle can be used to treat and/or prevent heart failure. Throughout this specification the words “prevent,” “preventing,” “prevention” and the like refer to delaying or forestalling the onset, development or progression of a condition or disease for a period of time, including weeks, months, or years. While current therapies for heart failure are aimed at delaying the progression of heart failure, rather than preventing the onset of heart failure, it is envisioned that the present technology can be employed to prevent the onset of heart failure in circumstances where subjects at risk of developing heart failure can be identified, e.g. by genetic testing or other means.

In another aspect, the heart failure is heart failure with preserved ejection fraction (HFpEF), also known as diastolic heart failure. In another aspect, the micelle can be used for treating and/or preventing heart failure in a subject who does not have and/or has not had a myocardial infarction or, more specifically, an acute myocardial infarction. In another aspect, the micelle can be used for treating and/or preventing heart failure in a subject who does not have and/or has not had cancer. In another aspect, the micelle can be used for treating and/or preventing heart failure in a subject who has been treated for cancer with certain drugs that, on occasion, may result in cardiac damage.

In yet another aspect, there is provided a micelle-forming amphiphilic block copolymer for carrying a cardioactive agent and localizing in fibrotic areas of the heart and more preferentially in areas in which cardiac fibroblasts are present. In another aspect, the amphiphilic block copolymer is as described above.

In yet another aspect, there is provided a micelle-forming amphiphilic block copolymer for delivering a cardioactive agent to fibrotic areas of the heart and more preferentially to areas in which cardiac fibroblasts are present. In another aspect, the amphiphilic block copolymer is as described above.

In yet another aspect, there is provided a composition comprising the micelle or micelle-forming amphiphilic block copolymer as described above. In yet another aspect, there is provided a drug delivery system comprising the micelle or micelle-forming amphiphilic block copolymer as described above. In another aspect, the system can be an implantable device.

In yet another aspect, there is provided a method for treating and/or preventing heart failure in a subject, the method comprising administering the micelle, micelle-forming amphiphilic block copolymer and/or composition as described above. In another aspect, the heart failure is diastolic heart failure, also known as HFpEF. In another aspect, the subject has cardiac arrhythmia.

In yet another aspect, there is provided a use of the micelle, micelle-forming amphiphilic block copolymer, composition and/or the drug delivery device as described above for treating and/or preventing heart failure in a subject. In another aspect, the heart failure is HFpEF. In another aspect, the subject has cardiac arrhythmia.

In yet another aspect, there is provided a method of passively targeting fibroblasts in the heart of a subject suffering from heart failure, the method comprising administering the micelle, micelle-forming amphiphilic block copolymer, and/or composition as described above. In another aspect, the heart failure is HFpEF. In another aspect, the subject has cardiac arrhythmia.

In yet another aspect, there is provided a use of the micelle, micelle-forming amphiphilic block copolymer, composition and/or the drug delivery device as described above for passively targeting fibroblasts in the heart of a subject suffering from heart failure. In another aspect, the heart failure is HFpEF. In another aspect, the subject has cardiac arrhythmia.

In yet another aspect, there is provided a method for treating and/or preventing cardiac arrhythmia in a subject, the method comprising administering the micelle, micelle-forming amphiphilic block copolymer and/or composition as described above.

In yet another aspect, there is provided a use of the micelle, micelle-forming amphiphilic block copolymer, composition and/or the drug delivery device as described above for treating and/or preventing cardiac arrhythmia in a subject.

In yet another aspect, there is provided a method of passively targeting fibroblasts in the heart of a subject suffering from cardiac arrhythmia, the method comprising administering the micelle, micelle-forming amphiphilic block copolymer, and/or composition as described above.

In yet another aspect, there is provided a use of the micelle, micelle-forming amphiphilic block copolymer, composition and/or the drug delivery device as described above for passively targeting fibroblasts in the heart of a subject suffering from cardiac arrhythmia.

It is understood that one or more of the aspects described herein (and above) may be combined in any suitable manner. The novel features of the present invention will become apparent to those of skill in the art upon examination of the following detailed description of the invention. It should be understood, however, that the specific examples presented, while indicating certain aspects of the present invention, are provided for illustration purposes only because various changes and modifications can be made thereto without departing from the scope of the invention herein described and claimed.

### BRIEF DESCRIPTION OF THE DRAWINGS

The present invention will be further understood from the following description with reference to the Figures, in which:

FIG. 1 shows fluorescent scans of whole mouse hearts removed from heart failure mice developed in a heart failure model 24 hours after administration of fluorescently-labelled nanoparticles by intravenous (i.v.) and intraperitoneal (i.p.) injection.

FIG. 2 shows fluorescence microscopy of fluorescently-labelled nanoparticles administered by subcutaneous (s.c.) injection in cardiac tissue of heart failure mice. (A) Differential interference contrast microscope image. (B) Fluorescence of Cy5.5 labelled nanoparticles. (C) Overlay of panels A and B.

FIG. 3 shows fluorescence microscopy of fluorescently-labelled nanoparticles in cardiac tissue of heart failure mice following administration by s.c. injection. (A) Fluorescence (light grey) of Cy5.5 labelled nanoparticles. (B) Overlay of 4,6-diamidino-2-phenylindole, dihydrochloride (DAPI) nuclear stain (dark grey) with Cy5.5 fluorescence (light grey). (C) Haemotoxylin and eosin (H&E) staining of the same area shown in A and B. (D) Enhanced overlay of A, B, and C.

FIG. 4 shows fluorescence microscopy of fluorescently-labelled nanoparticles in cardiac tissue of heart failure mice following administration by s.c. injection. (A) Fluorescence (light grey) of Cy5.5 labelled nanoparticles. (B) Overlay of DAPI nuclear stain (dark grey) with Cy5.5 fluorescence (light grey). (C) H&E staining of the same area shown in A and B. (D) Enhanced overlay of A, B, and C.

FIG. 5 shows fluorescence microscopy of fluorescently-labelled nanoparticles in cardiac tissue of heart failure mice after administration by s.c. injection. (A) Overlay of Cy5.5 label (light grey) and DAPI stained nuclei (dark grey). (B) Enlargement of panel A.

FIG. 6 is a bar graph showing the normalized diameter of myocytes from control mice hearts and hearts from mice treated with angiotensin II alone, angiotensin II in combination with free cyclosporine A, and angiotensin II in combination with cyclosporine A encapsulated in micelles formed from a block copolymer, PEG-PCL.

FIG. 7 is a bar graph showing the degree of B-type Natriuretic Peptide (BNP) mRNA expression in the hearts of control mice compared to mice treated with angiotensin II alone, angiotensin II in combination with free cyclosporine A, and angiotensin II in combination with cyclosporine A encapsulated in micelles formed from a block copolymer, PEG-PCL.

FIG. 8 is a graph showing the pharmacokinetic profile of free versus encapsulated CBD administered subcutaneously over a 72-hour period.

### DETAILED DESCRIPTION

### Definitions

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Although any methods and materials similar or equivalent to those described herein can be used in the practice for testing of the present invention, the typical materials and methods are described herein. In describing and claiming the present invention, the following terminology will be used.

It is also to be understood that the terminology used herein is for the purpose of describing aspects only and is not intended to be limiting. Many patent applications, patents, and publications are cited herein to assist in understanding the aspects described. All such references cited herein are incorporated herein by reference in their entirety and for all purposes to the same extent as if each individual publication or patent or patent application was specifically and individually indicated to be incorporated by reference in its entirety for all purposes. To the extent publications and patents or patent applications incorporated by reference contradict the disclosure contained in the specification, the specification is intended to supersede and/or take precedence over any such contradictory material.

In understanding the scope of the present application, the articles “a”, “an”, “the”, and “said” are intended to mean that there are one or more of the elements. Additionally, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

It will be understood that any aspects described as “comprising” certain components may also “consist of” or “consist essentially of,” wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Typically, a composition consisting essentially of a set of components will comprise less than 5% by weight, typically less than 3% by weight, more typically less than 1%, and even more typically less than 0.1% by weight of non-specified component(s).

It will be understood that any component defined herein as being included may be explicitly excluded from the claimed invention by way of proviso or negative limitation. For example, in certain aspects, any cardioactive agents listed herein may be excluded, such as methotrexate. In additional or alternative aspects, any polymers may be excluded, such as one or more of those described in U.S. Pat. No. 8,309,515 or 9,139,553. In some aspects, the micelle will not be housed within a suitable carrier or, in other words, it will be naked and will be administered systemically in this manner (i.e., without a carrier). In other aspects, the subject does not have and/or has not had a myocardial infarction or, more specifically, an acute myocardial infarction.

In addition, all ranges given herein include the end of the ranges and any intermediate range points, whether explicitly stated or not.

Terms of degree such as “substantially”, “about” and “approximately” as used herein mean a reasonable amount of deviation of the modified term such that the result is not significantly changed. These terms of degree should be construed as including a deviation of up to ±5% of the modified term if this deviation would not negate the meaning of the word it modifies.

The abbreviation, “e.g.” is derived from the Latin exempli gratia, and is used herein to indicate a non-limiting example. Thus, the abbreviation “e.g.” is synonymous with the term “for example.” The word “or” is intended to include “and” unless the context clearly indicates otherwise.

It is further to be understood that all micelle sizes, and all molecular weight or molecular mass values, are approximate and are provided for description. Although methods and materials similar or equivalent to those described herein can be used in the practice or testing of this disclosure, suitable methods and materials are described below.

“Active” or “activity” for the purposes herein refers to a biological activity of the compositions described herein, wherein “biological” activity refers to a biological function (either inhibitory or stimulatory) caused by the compositions.

Administration “in combination with” one or more further therapeutic agents includes simultaneous (concurrent) and consecutive administration in any order.

“Amelioration” means a lessening of severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

“Amphiphilic block copolymer” as used herein encompasses block copolymers such as di-block copolymers as well as tri-block copolymers, wherein at least one polymeric block is hydrophilic and at least one polymeric block is hydrophobic. In this way, the amphiphilic block copolymers can assemble, either through self-assembly or assisted-assembly, into a micellar structure (which includes a vesicular structure). In some aspects, the micelle comprising the amphiphilic block copolymer exhibits a molecular weight of greater than about 3000 Daltons. In a particular aspect, the molecular weight of the copolymer is between about 3,000-about 50,000 Daltons. In one aspect, the hydrophilic block may have a number average molecular weight of from about 200 to about 29,000 daltons, greater than about 2,000 daltons, greater than about 3,000 daltons, or greater than about 5000 daltons, or, typically, from about 2000 to about 20,000 daltons. The hydrophilic block may be one or more hydrophilic polymers selected from the group consisting of polyalkyleneglycol (PAG), polyacrylic acid (PAA), polyacrylonitrile (PAN), polyethyleneoxide (PEO), polyvinylacetate (PVAc), polyethyleneglycol (PEG), polyvinylpyrrolidone (PVP), polyacrylamide, polyvinylalcohol (PVA) and hydrophilic poly(amino acid)s. For example, the hydrophilic polymer may be one or more selected from the group consisting of (mono)methoxypolyethylene glycol, (mono)acetoxypolyethylene glycol, polyethylene glycol, a copolymer of polyethylene and propylene glycol, polyvinylpyrrolidone, poly(glutamine), polyglutamic acid, polythreonine, poly(asparagine), poly(arginine) and poly(serine). The hydrophilic polymer also includes a derivative thereof.

In one aspect, any hydrophobic polymer may be used if it is a material capable of forming an amphiphilic block copolymer in combination with a hydrophilic polymer. In one aspect, the hydrophobic block may have a number average molecular weight of from about 200 to about 29,000 daltons, greater than about 2,000 daltons, greater than about 3,000 daltons, or greater than about 5000 daltons, or, typically, from about 2000 to about 20,000 daltons The hydrophobic polymer also includes a derivative thereof. The hydrophobic block may be one or more hydrophobic polymers selected from the group consisting of polyester, poly(anhydride), hydrophobic poly(amino acid), polyorthoester and polyphosphazene. The hydrophobic block is typically one or more selected from the group consisting of polyleucine, polyisoleucine, polyvaline, polyphenylalanine, polyproline, polyglycine and polymethionine, polytryptophane, polyalanine, polylactide, polyglycolide, polycaprolactone, polydioxane-2-one, a copolymer of polylactide and glycolide, a copolymer of polylactide and dioxane-2-one, a copolymer of polylactide and caprolactone, and a copolymer of polyglycolide and caprolactone.

The hydrophobic block encompasses a lipophilic compound and therefore, may also be a “lipid” or “lipid polymer,” which, as used herein encompasses phospholipids, lipid proteins, glycolipids, and cationic lipids if they are able to form a micellar structure. Also, the lipid encompasses a naturally-induced lipid and a synthetic lipid derivative. The phospholipids include glycerophospholipids and phosphosphingolipids. The glycerophospholipids may include a diacylglyceride structure and specifically include phosphatidic acid (PA), lecithin (phosphatidylcholine, PC), cephalin and phosphoinositides. The cephalin phospholipids include phosphatidylserine (PS) and phosphatidylethanolamine (PE). Also, the phosphoinositide-like phospholipids include phosphatidylinositol (PI), phosphatidylinositol phosphate (PIP), phosphatidylinositol bisphosphate (PIP2) and phosphatidylinositol triphosphate (PIP3). The sphingophospholipids include ceramide phosphorylcholine (sphingomyelin, SPH), ceramide phosphorylethanolamine (sphingomyelin, Cer-PE) and ceramide phosphoryllipid. There is no limit to the type of synthetic phospholipid derivative, but in one aspect, the synthetic phospholipid derivative may be selected from the group consisting of 1,2-didodecanoyl-sn-glycero-3-ethylphosphocholine (EPC), 11,2-dioleoyl-sn-glycero-3-phosphocholine (DOPC), 1-palmitoyl-2-oleoyl-sn-glycero-3-phosphoethanolamine (POPE), 1-palmitoyl-2-oleoyl-sn-glycero-3-phospho-L-serine (POPS), 1,2-distearoyl-sn-glycero-3-phosphoethanolamine (DSPE) and combinations thereof.

Examples of amphiphilic block copolymers suitable for use herein are typically biocompatible and biodegradable and include, for example, a) PEO-poly(ester)s, such as, and without being limited thereto, PEO-polycaprolactone, PEO-poly(valerolactone), PEO-poly(butyrolactone), PEO-polylactides, PEO-polyglycolides, PEO-polylactide-glycolide or a mixtures thereof, or blocks with random poly(ester)s, and their derivatives; b) PEO-poly(amino acid)s, such as, and without being limited thereto, PEO-poly(aspartic acid); PEO-poly(glutamic acid, typically polymers of either of 20 natural amino acids, or their random mixture, or polymers of derivatives (includes analogues) of natural amino acids; c) PEO-phospholipids, such as, and without being limited thereto, 1,2-distearoyl-sn-glycero-3-phosphoethanolamine-N-[amino(polyethylene glycol) (PEG-DSPE). In addition, any of the above amphiphilic block copolymers may have PEO replaced with PVP. In another aspect, the amphiphilic block copolymer comprises a hydrophilic block selected from the group consisting of PEO, PVP, derivatives thereof, and combinations thereof. In another aspect, the amphiphilic block copolymer comprises a hydrophobic block selected from the group consisting of a poly(ester), a poly(amino acid), a phospholipid, derivatives thereof, and combinations thereof. In another aspect, the amphiphilic block copolymer is selected from the group consisting of PEO-polycaprolactone, PEO-poly(valerolactone), PEO-poly(butyrolactone)s, PEO-polylactones, PEO-polylactides, PEO-polyglycolides, PEO-polylactide-glycolide, PEO-poly(aspartic acid), PEO-poly(glutamic acid), 1,2-distearoyl-sn-glycero-3-phosphoethanolamine-N-[amino(polyethylene glycol) (PEG-DSPE), poly(ethylene oxide)-poly(caprolactone) (PEO-PCL), poly(ethylene oxide)-block-poly(α-benzyl carboxylate-ε-caprolactone) (PEO-PBCL), poly(ethylene oxide)-block-poly(α-carboxylate-ε-caprolactone) (PEO-PCCL), poly(ethylene oxide)-block-poly(α-cholestryl carboxylate-ε-caprolactone) (PEO-PChCL), derivatives thereof, and combinations thereof. In another example, wherein the amphiphilic block copolymer is poly(ethylene oxide)-poly(caprolactone) (PEO-PCL), poly(ethylene oxide)-block-poly(α-benzyl carboxylate-ε-caprolactone) (PEO-PBCL), poly(ethylene oxide)-block-poly(α-carboxylate-ε-caprolactone) (PEO-PCCL), derivatives thereof or combinations thereof. In another example, wherein the amphiphilic block copolymer is poly(ethylene oxide)-poly(caprolactone) (PEO-PCL), poly(ethylene oxide)-block-poly(α-benzyl carboxylate-ε-caprolactone) (PEO-PBCL), poly(ethylene oxide)-block-poly(α-carboxylate-ε-caprolactone) (PEO-PCCL), poly(ethylene oxide)-poly(caprolactone)-poly(α-propargyl carboxylate-ε-caprolactone) (PEO-PCL-PCC), poly(ethylene oxide)-block-poly(α-benzyl carboxylate-ε-caprolactone)-poly(α-propargyl carboxylate-ε-caprolactone) (PEO-PBCL-PCC), poly(ethylene oxide)-block-poly(α-carboxylate-ε-caprolactone)-poly(α-propargyl carboxylate-ε-caprolactone) (PEO-PCCL-PCC), derivatives thereof or combinations thereof. In another aspect, the amphiphilic block copolymer is PEOₙ-PBCLₘ where n can be 10-300, 50-250, 75-200, 75-150, 100-150, or 100-125 and m can be 5-200, 5-150, 5-100, 10-100, 10-50, 10-30, 10-25 or 20-25. In another aspect, the amphiphilic block copolymer is PEOₙ-PCLₘ where n can be 10-300, 50-250, 75-200, 75-150, 100-150, or 100-125 and m can be 5-200, 5-150, 5-100, 10-100, 10-50, 10-30, 10-25 or 20-25. In another aspect, the amphiphilic block copolymer is PEOₙ-PCCLₘ where n can be 10-300, 50-250, 75-200, 75-150, 100-150, or 100-125 and m can be 5-200, 5-150, 5-100, 10-100, 10-50, 10-30, 10-25 or 20-25. With respect to the nomenclature regarding “-block-”, “-b-”, or “-”, these are used interchangeably.

“Biodegradable” means the conversion of materials into less complex intermediates or end products by solubilization hydrolysis, or by the action of biologically formed entities which can be enzymes and other products of the organism.

“Biocompatible” means materials or the intermediates or end products of materials formed by solubilization hydrolysis, or by the action of biologically formed entities which can be enzymes and other products of the organism and which cause no adverse effects to the body.

“Block copolymer” means a polymer whose molecule consists of blocks of different species of polymers that are connected linearly.

“Cardioactive agent” refers to any bioactive agent or class of bioactive agents that can be used in the treatment and/or prevention of a heart-related condition or disease such as a fibrotic and/or inflammatory condition, particularly heart failure as described herein. These may be any known agents. Cardioactive agents include, but are not limited to, anti-fibrotic agents, anti-inflammatory agents, statins, nitrates, beta-blockers, TLR4 antagonists, any blockers of HSP60 activity or inhibitors of production and/or transport of HSP 60, diuretics, inotropes, digoxin, vasodilators, angiotensin II converting enzyme (ACE) inhibitors, angiotensin II receptor blockers (ARB), sacubitril/valsarten, soluble guanylate cyclase modulators, omecamtiv mecarbil, tacrolimus, calcium channel blockers, hydralazine, natriuretic peptides, and cannabinoids. The term “anti-fibrotic agent,” as used herein, includes any agent that reduces or treats fibrosis, including, but not limited to, an anti-angiogenic agent, a vascular endothelial growth factor (VEGF) antagonist, a basic fibroblast growth factor (bFGF) antagonist, a bFGF receptor antagonist, a transforming growth factor-beta (TGF-β) antagonist, a TGF-β receptor antagonist, a steroidal anti-inflammatory agent, and a non-pirfenidone TNF antagonist. The term “non-pirfenidone TNF-α antagonist,” as used herein, refers to tumor necrosis factor (TNF) antagonists, such as anti-TNF antibodies (e.g. REMICADE™ anti-TNF monoclonal antibody) and soluble TNF receptor (e.g. ENBREL™ TNF receptor-Ig immunoadhesin), and HUMIRA®. The terms “angiogenic agent,” “angiogenic compound,” and “angiogenic factor” are meant to include agents that promote neovascularization, such as VEGF, bFGF, and TGF-beta. The terms “anti-angiogenic” or “angiostatic” agent, drug or compound, or “angiogenesis inhibitor,” are meant to include agents that prevent or reduce neovascularization, such as VEGF antagonists, VEGF receptor antagonists, bFGF antagonists, bFGF receptor antagonists, TGF-beta antagonists, and TGF-beta receptor antagonists.

Other specific examples of such cardioactive agents include but are not limited to, sildenafil, pirfenidone, rapamycin, methotrexate, amiodarone, cyclosporine, cyclosporine A, cyclosporine D, valspodar, spironolactone, eplerenone, furosemide, dobutamine, milrinone, captopril, enalapril, lisinopril, benazepril, quinapril, fosinopril, ramipril, candesartan, irbesartan, olmesartan, losartan, valsartan, telmisartan, eprosartan, isosorbide mononitrate, isosorbide dinitrate, carvedilol, metoprolol, nesiritide, thalidomide, cannabidiol, and any derivatives thereof.

“Critical micelle concentration (CMC)” refers to the concentration above which amphiphilic molecules including block copolymers self-assemble and form a supramolecular core/shell structure, i.e., a micelle.

The terms “fibrotic condition,” “fibroproliferative condition,” “fibrotic disease,” “fibroproliferative disease,” “fibrotic disorder,” and “fibroproliferative disorder” are used interchangeably to refer to a condition, disease or disorder that is characterized by dysregulated proliferation or activity of fibroblasts and/or pathologic or excessive accumulation of collagenous tissue. Typically, any such disease, disorder or condition is amenable to treatment by administration of a compound having anti-fibrotic activity. Fibrosis is generally characterized by the pathologic or excessive accumulation of collagenous connective tissue. Fibrotic disorders include, but are not limited to, collagen disease, interstitial lung disease, human fibrotic lung disease (e.g., obliterative bronchiolitis, idiopathic pulmonary fibrosis, pulmonary fibrosis from a known etiology, tumor stroma in lung disease, systemic sclerosis affecting the lungs, Hermansky-Pudlak syndrome, coal worker's pneumoconiosis, asbestosis, silicosis, chronic pulmonary hypertension, AIDS-associated pulmonary hypertension, sarcoidosis, and the like), fibrotic vascular disease, arterial sclerosis, atherosclerosis, varicose veins, myocardial infarcts, cerebral infarcts, myocardial fibrosis, musculoskeletal fibrosis, post-surgical adhesions, human kidney disease (e.g., nephritic syndrome, Alport's syndrome, HIV-associated nephropathy, polycystic kidney disease, Fabry's disease, diabetic nephropathy, chronic glomerulonephritis, nephritis associated with systemic lupus, and the like), cutis keloid formation, progressive systemic sclerosis (PSS), primary sclerosing cholangitis (PSC), liver fibrosis, liver cirrhosis, renal fibrosis, pulmonary fibrosis, cystic fibrosis, chronic graft versus host disease, scleroderma (local and systemic), Grave's ophthalmopathy, diabetic retinopathy, glaucoma, Peyronie's disease, penis fibrosis, urethrostenosis after the test using a cystoscope, inner accretion after surgery, scarring, myelofibrosis, idiopathic retroperitoneal fibrosis, peritoneal fibrosis from a known etiology, drug-induced ergotism, fibrosis incident to benign or malignant cancer, fibrosis incident to microbial infection (e.g., viral, bacterial, parasitic, fungal, etc.), Alzheimer's disease, fibrosis incident to inflammatory bowel disease (including stricture formation in Crohn's disease and microscopic colitis), fibrosis induced by chemical or environmental insult (e.g., cancer chemotherapy, pesticides, radiation (e.g., cancer radiotherapy and the like), and the like. Neuroinflammatory conditions and other inflammatory conditions, such as rheumatoid arthritis, are also included herein. Typically, the fibrotic condition described herein is heart-related and, more typically, is heart failure.

“Fibrosis” means the formation or development of excess fibrous connective tissue in an organ or tissue. In certain embodiments, fibrosis occurs as a reparative or reactive process. In certain embodiments, fibrosis occurs in response to damage or injury. The term “fibrosis” is to be understood as the formation or development of excess fibrous connective tissue in an organ or tissue as a reparative or reactive process, as opposed to a formation of fibrous tissue as a normal constituent of an organ or tissue, and is frequently caused by the presence of inflammation.

The term “micelle” is used herein according to its art-recognized meaning and as well includes all forms of micelles, including, for example, spherical micelles, cylindrical micelles, worm-like micelles and sheet-like micelles, and vesicles, formed in water, or mostly water. The micelles described herein are typically of a size selected to localize to areas in the heart where cardiac fibroblasts are present when administered systemically. The micelles can have a size ranging from about 10 nm, about 25 nm, about 50 nm, about 75 nm, about 100 nm, about 125 nm, about 150 nm, or about 175 nm and/or up to about 500 nm, about 400 nm, about 300 nm, about 250 nm, or about 200 nm.

It will be understood that these sizes refer to the diameter of a spherical micelle or the smallest width of a non-spherical micelle. For example, a worm-like micelle may have a width of up to about 500 nm, as described above. However, its length is not restricted in this way and can be up to a micron or more. In some aspects, worm-like micelles are formed from aggregates of other types of micelles, such as spherical micelles, aggregated substantially linearly to form a worm-like structure. These worm-like micelles may release the aggregated micelles from the worm-like structure slowly over time leading to an increased half-life.

According to an embodiment, the micelle may be prepared by a known method without limitation. For example, the micelle may be prepared by a method of dispersing an amphiphilic block copolymer including a hydrophilic domain and a hydrophobic domain in an aqueous solution and performing sonication, a method of dispersing or dissolving an amphiphilic block copolymer including a hydrophilic domain and a hydrophobic domain in an organic solvent and extracting or evaporating the organic solvent with an excess amount of water, a method of dialyzing an organic solvent with an excess amount of water after dispersion or dissolution of an amphiphilic block copolymer including a hydrophilic domain and a hydrophobic domain in an organic solvent, a method of dispersing or dissolving an amphiphilic block copolymer including a hydrophilic domain and a hydrophobic domain in an organic solvent and vigorously evaporating the solvent using a homogenizer or a high pressure emulsifier, thin film hydration, or the like.

“Modulating” means mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, typically, a human.

“Molecular weight” (M_{w}) means average molecular weight and the units can be in Daltons or g/mol.

“Parenteral” administration includes, e.g., subcutaneous (s.c.), intravenous (i.v.), intramuscular (i.m.), intraperitoneal (i.p.), or intrasternal injection, infusion techniques, or absorption through mucous membranes.

The terms “PEG” and “PEO” are used interchangeably herein and it will be understood that these are polymers derived from the same monomers. Materials with a M_{w}<20,000 are usually called PEGs, while higher molecular weight polymers are classified as PEOs. To avoid any confusion, the use of PEG or PEO herein is not intended to provide any inference with respect to molecular weight; these terms are used completely interchangeably and desired molecular weights will be specified separately.

“Pharmaceutically acceptable” means that the compound or combination of compounds is compatible with the remaining ingredients of a formulation for pharmaceutical use, and that it is generally safe for administering to humans according to established governmental standards, including those promulgated by the United States Food and Drug Administration.

“Pharmaceutically acceptable carrier” includes, but is not limited to solvents, dispersion media, antibacterial agents, antifungal agents, isotonic and/or absorption delaying agents and the like. The use of pharmaceutically acceptable carriers is well known.

The words “Preventing,” “prevent,” “prevention,” and the like, refer to delaying or forestalling the onset, development or progression of a condition or disease for a period of time, including weeks, months, or years. While current therapies for heart failure are aimed at delaying the progression of heart failure, rather than preventing the onset of heart failure, it is envisioned that the present technology can be employed to prevent the onset of heart failure in future circumstances where subjects at risk of developing heart failure can be identified, e.g. by genetic testing or other means.

“Subject suspected of having” means a subject exhibiting one or more clinical indicators of a disease or condition, such as fibrosis, heart inflammation, and/or heart failure.

The terms “therapeutically effective amount”, “effective amount” or “sufficient amount” mean a quantity sufficient, when administered to a subject, including a mammal, for example a human, to achieve a desired result, for example an amount effective to cause a protective immune response. Effective amounts of the compounds described herein may vary according to factors such as the immunogen, age, sex, and weight of the subject. Dosage or treatment regimes may be adjusted to provide the optimum therapeutic response, as is understood by a skilled person. For example, administration of a therapeutically effective amount of the composition described herein is, in aspects, sufficient to treat and/or prevent heart failure in a subject.

Moreover, a treatment regime of a subject with a therapeutically effective amount may consist of a single administration, or alternatively comprise a series of applications. The length of the treatment period depends on a variety of factors, such as the polymers used to make the micelles, the cardioactive agent used in conjunction with the micelles, the age of the subject, the concentration of the cardioactive agent, the responsiveness of the patient to the cardioactive agent, or a combination thereof. It will also be appreciated that the effective dosage of the cardioactive agent used for the treatment may increase or decrease over the course of a particular treatment regime. Changes in dosage may result and become apparent by standard diagnostic assays known in the art. The compositions described herein may, in aspects, be administered before, during, or after treatment with conventional therapies for heart failure.

In some aspects, effective amounts of a cardioactive agent are amounts that, in monotherapy or combination therapy, when administered to an individual having heart failure is effective to reduce the rate of progression of fibrosis by at least about 10%, at least about 15%, at least about 20%, at least about 25%, at least about 30%, at least about 35%, at least about 40%, at least about 45%, or at least about 50%, or more, compared to the rate of progression of fibrosis that would have been experienced by the patient in the absence of the cardioactive monotherapy or combination therapy.

In some aspects, effective amounts of a cardioactive agent are amounts that, in monotherapy or combination therapy, when administered to an individual having heart failure are effective to reduce the rate of deterioration of at least one function of the heart by at least about 10%, at least about 15%, at least about 20%, at least about 25%, at least about 30%, at least about 35%, at least about 40%, at least about 45%, or at least about 50%, or more, compared to the rate of deterioration of heart function that would have been experienced by the individual in the absence of the subject monotherapy or combination therapy.

Methods of measuring the extent of fibrosis and inflammation of the heart, and methods of measuring the function of the heart are known.

“Treatment,” “treating,” or “treat,” and the like, means the application of one or more specific procedures used for curing or ameliorating a disease. In certain embodiments, the specific procedure is the administration of one or more pharmaceutical agents.

“Subject” means any member of the animal kingdom, typically a mammal. The term “mammal” refers to any animal classified as a mammal, including humans, other higher primates, domestic and farm animals, and zoo, sports, or pet animals, such as dogs, cats, cattle, horses, sheep, pigs, goats, rabbits, etc. Typically, the mammal is human.

“Water-insoluble” means molecules or materials which are incapable or poorly capable of dissolving in water; for example, a drug that precipitates at concentrations greater than 10 mg/ml in water is considered to be “water-insoluble.”

“Water miscible organic solvent” means organic solvents that can be mixed with water and form one phase (not separated) such as acetonitrile, ethyl acetate, methanol, ethanol, propylene glycol, tetrahydrofuran (THF), etc.

The term “C₁₋₂₀ alkyl” as used herein means straight and/or branched chain alkyl groups containing from one to twenty carbon atoms and includes methyl, ethyl, propyl, isopropyl, t-butyl, pentyl, hexyl and the like.

The term “C₃₋₂₀ cycloalkyl” as used herein means saturated cyclic alkyl radicals containing from three to twenty carbon atoms and includes cyclopropyl, cyclobutyl, cyclopentyl, cyclohexyl, cycloheptyl and the like.

The term “aryl” as used herein means a monocyclic or bicyclic carbocyclic ring system containing one or more aromatic rings, in particular embodiments, one or two aromatic rings and from 6 to 14 carbon atoms and includes phenyl, benzyl, naphthyl, anthraceneyl, 1,2-dihydronaphthyl, 1,2,3,4-tetrahydronaphthyl, fluorenyl, indanyl, indenyl and the like.

The term “C₂₋₆ alkenyl” as used herein means straight and/or branched chain alkenyl groups containing from two to six carbon atoms and one to three double bonds and includes vinyl, allyl, 1-butenyl, 2-hexenyl and the like.

The term “C₂₋₆ alkenyloxy” as used herein means straight and/or branched chain alkenyloxy groups containing from two to six carbon atoms and one to three double bonds and includes vinyloxy, allyloxy, propenyloxyl, butenyloxy, hexenyloxy and the like.

The term “alkylene” as used herein means bifunctional straight and/or branched alkyl radicals containing the specified number of carbon atoms.

The term “halo” as used herein means halogen and includes chloro, fluoro, bromo, iodo and the like.

### Amphiphilic Block Copolymers, Micelles, and Compositions

Described herein are amphiphilic block copolymers. These amphiphilic block copolymers typically self-assemble into micelles. One class of suitable amphiphilic block copolymers for use herein are described in U.S. Pat. Nos. 8,309,515 and 9,139,553, the disclosures of which are incorporated herein by reference in their entireties. These patents describe micelle-forming poly(ethylene oxide)-block-poly(ester) block copolymers having reactive groups on the polyester block therein, which have now been found to be particularly well-suited to the delivery of cardioactive agents to cardiac tissue. The present inventors have found, surprisingly, that the block copolymers described herein preferentially accumulate in heart tissue, more preferentially accumulate in inflamed heart tissue, and still more preferentially accumulate in the area where fibroblasts exist in a failing heart, in a murine model of heart failure. Methods of synthesis for these copolymers are also described in U.S. Pat. Nos. 8,309,515 and 9,139,553.

Thus, in a specific aspect, the amphiphilic block copolymer for use herein comprises a compound of the formula I:

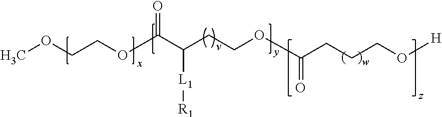

wherein

L₁ is a linker group selected from the group consisting of a single bond, —C(O)—O—, —C(O)— and —C(O)NR²;

R₁ is selected from the group consisting of H, OH, C₁₋₂₀ alkyl, C₃₋₂₀ cycloalkyl and aryl, said latter three groups may be optionally substituted and in which one or more of the carbons of the alkyl, cycloalkyl or aryl groups may optionally be replaced with O, S, N, NR² or N(R²)₂ or R₁ is a bioactive agent;

R² is H or C₁₋₆alkyl;

v and w are independently of each other, an integer independently selected from 1 to 4;

x is an integer from 10 to 300;

y is an integer from 5 to 200;

z is an integer from 0 to 100;

wherein aryl is a mono- or bicyclic aromatic radical containing from 6 to 14 carbon atoms having a single ring or multiple condensed rings; and

wherein the optional substituents are selected from the group consisting of halo, OH, OC₁₋₆ alkyl, C₁₋₆ alkyl, C₂₋₆ alkenyl, C₂₋₆ alkenyloxy, NH₂, NH(C₁₋₆ alkyl), N(C₁₋₆ alkyl)(C₁₋₆ alkyl), CN, NO₂, C(O)C₁₋₆ alkyl, C(O)OC₁₋₆ alkyl, SO₂C₁₋₆ alkyl, SO₂NH₂, SO₂NHC₁₋₆ alkyl, phenyl and C₁₋₆ alkylenephenyl.

In one aspect, L₁ is —C(O)—O— or —C(O)—. In a further aspect, R₁ is selected from the group consisting of optionally substituted C₁₋₆ alkyl, C₃₋₈ cycloalkyl, aryl in which one or more of the carbons of the alkyl, cycloalkyl or aryl groups may optionally be replaced with O, S or N, and a bioactive agent. In a further aspect, the bioactive agent is a cardioactive agent, such as a drug useful to treat or prevent heart failure, such as cyclosporine A or cannabidiol.

In an aspect, the optional substituents are selected from the group consisting of halo, OH, OC₁₋₄ alkoxy, C₁₋₄ alkyl, C₂₋₄ alkenyl, C₂₋₄ alkenyloxy, NH₂, NH(C₁₋₄ alkyl), N(C₁₋₄ alkyl)(C₁₋₄ alkyl), CN, NO₂, C(O)C₁₋₄ alkyl, C(O)OC₁₋₄ alkyl, SO₂C₁₋₄ alkyl, SO₂NH₂, SO₂NHC₁₋₄ alkyl, phenyl and C₁₋₄ alkylenephenyl.

In yet another aspect, v and w are, independently of each other, 2 or 3.

In yet another aspect, v and w are equal.

In another aspect, x is an integer from 50 to 200. In a more particular aspect, x is an integer from 100 to 150.

In another aspect, y is an integer from 5 to 100. In a more particular aspect, y is an integer from 5 to 50. In an even more particular aspect, y is an integer from 10 to 20.

In an aspect, z is an integer from 0 to 80, more suitably from 0 to 40.

In another aspect, R₁ is a bioactive agent. In a further aspect, the bioactive agent is a cardioactive agent, such as a drug useful to treat or prevent heart failure, such as cyclosporine A or cannabidiol.

In another specific aspect, the amphiphilic block copolymer for use herein comprises a compound of the formula I:

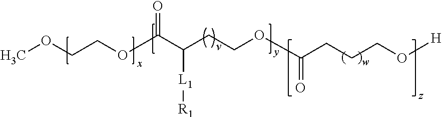

wherein

L₁ is a linker group selected from the group consisting of a single bond, —C(O)—O—, —C(O)—, —O—, —S—, —NH—, —NR²—, and —C(O)NR²;

R₁ is selected from the group consisting of H, OH, C₁₋₂₀ alkyl, C₃₋₂₀ cycloalkyl and aryl, said latter three groups may be optionally substituted and in which one or more of the carbons of the alkyl, cycloalkyl or aryl groups may optionally be replaced with O, S, N, NR² or N(R²)₂ or R₁ is a bioactive agent;

R² is H or C₁₋₆alkyl;

v and w are independently of each other, an integer independently selected from 1 to 4;

x is an integer from 10 to 300;

y is an integer from 5 to 200;

z is an integer from 0 to 100;

wherein aryl is a mono- or bicyclic aromatic radical containing from 6 to 14 carbon atoms having a single ring or multiple condensed rings; and

wherein the optional substituents are selected from the group consisting of halo, OH, OC₁₋₆ alkyl, C₁₋₆ alkyl, C₂₋₆ alkenyl, C₂₋₆ alkenyloxy, NH₂, NH(C₁₋₆ alkyl), N(C₁₋₆ alkyl)(C₁₋₆ alkyl), CN, NO₂, C(O)C₁₋₆ alkyl, C(O)OC₁₋₆ alkyl, SO₂C₁₋₆ alkyl, SO₂NH₂, SO₂NHC₁₋₆ alkyl, phenyl and C₁₋₆ alkylenephenyl.

In one aspect, L₁ is —C(O)—O— or —C(O)—. In a further aspect, R₁ is selected from the group consisting of optionally substituted C₁₋₆ alkyl, C₃₋₈ cycloalkyl, aryl in which one or more of the carbons of the alkyl, cycloalkyl or aryl groups may optionally be replaced with O, S or N, and a bioactive agent. In a further aspect, the bioactive agent is a cardioactive agent, such as a drug useful to treat or prevent heart failure, such as cyclosporine A or cannabidiol.

In an aspect, the optional substituents are selected from the group consisting of halo, OH, OC₁₋₄ alkoxy, C₁₋₄ alkyl, C₂₋₄ alkenyl, C₂₋₄ alkenyloxy, NH₂, NH(C₁₋₄ alkyl), N(C₁₋₄ alkyl)(C₁₋₄ alkyl), CN, NO₂, C(O)C₁₋₄ alkyl, C(O)OC₁₋₄ alkyl, SO₂C₁₋₄ alkyl, SO₂NH₂, SO₂NHC₁₋₄ alkyl, phenyl and C₁₋₄ alkylenephenyl.

In yet another aspect, v and w are, independently of each other, 2 or 3.

In yet another aspect, v and w are equal.

In another aspect, x is an integer from 50 to 200. In a more particular aspect, x is an integer from 100 to 150.

In another aspect, y is an integer from 5 to 100. In a more particular aspect, y is an integer from 5 to 50. In an even more particular aspect, y is an integer from 10 to 20.

In an aspect, z is an integer from 0 to 80, more suitably from 0 to 40.

In another aspect, R₁ is a bioactive agent. In a further aspect, the bioactive agent is a cardioactive agent, such as a drug useful to treat or prevent heart failure, such as cyclosporine A or cannabidiol.

In another specific aspect, the amphiphilic block copolymer for use herein comprises a compound of the formula I:

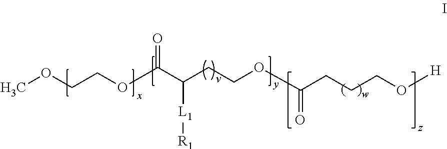

wherein

L₁ is a linker group selected from the group consisting of the following: a single bond, —C(O)—O—, —C(O)— and —C(O)NR²;

R₁ is selected from the group consisting of OH, C₃₋₂₀ cycloalkyl and aryl, said latter two groups may be optionally substituted and in which one or more of the carbons of the alkyl, cycloalkyl or aryl groups may optionally be replaced with O, S, N, NR² or N(R²)₂ or R₁ is a bioactive agent;

R² is H or C₁₋₆ alkyl;

v and w are, independently of each other, an integer independently selected from 1 to 4;

x is an integer from 10 to 300;

y is an integer from 5 to 200;

z is an integer from 0 to 100;

wherein aryl is mono- or bicyclic aromatic radical containing from 6 to 14 carbon atoms having a single ring or multiple condensed rings; and wherein the optional substituents are selected from the group consisting of halo, OH, OC₁₋₆ alkyl, C₁₋₆ alkyl, C₂₋₆ alkenyl, C₂₋₆ alkenyloxy, NH₂, NH(C₁₋₆ alkyl), N(C₁₋₆alkyl)(C₁₋₆ alkyl), CN, NO₂, C(O)C₁₋₆ alkyl, C(O)OC₁₋₆ alkyl, SO₂C₁₋₆ alkyl, SO₂NH₂, SO₂NHC₁₋₆ alkyl, phenyl and C₁₋₆ alkylenephenyl.

In another aspect, L₁ is —C(O)—O— or —C(O)—.

In a further aspect, the optional substituents are selected from the group consisting of halo, OH, OC₁₋₄ alkoxy, C₁₋₄ alkyl, C₂₋₄ alkenyl, C₂₋₄ alkenyloxy, NH₂, NH(C₁₋₄ alkyl), N(C₁₋₄ alkyl)(C₁₋₄ alkyl), CN, NO₂, C(O)C₁₋₄ alkyl, C(O)OC₁₋₄ alkyl, SO₂C₁₋₄ alkyl, SO₂NH₂, SO₂NHC₁₋₄ alkyl, phenyl and C₁₋₄ alkylenephenyl.

In another aspect, v and w are, independently of each other, 2 or 3.

In another aspect, v and w are equal.

In another aspect, x is an integer from 50 to 200.

In another aspect, y is an integer from 5 to 100.

In another aspect, z is an integer from 0 to 80.

In another aspect, R₁ is a bioactive agent. In a further aspect, the bioactive agent is a cardioactive agent, such as a drug useful to treat or prevent heart failure, such as cyclosporine A or cannabidiol.

Another class of suitable amphiphilic block copolymers for use herein are described in International Patent Application No. WO 2005/118672, which is incorporated herein by reference in its entirety. The Lavasanifar group has further published on micellar structures carrying bioactive agents. See, for example, International Patent Application Publication No. WO 2005/118672; Hamdy et al. (The AAPS Journal, 2011, 13(2):159-168); Binkhathlan et al. (Current Drug Delivery, 2012, 9:164-171); Aliabadi et al. (Biomaterials, 2005, 26:7251-7259); Aliabadi et al. (Journal of Controlled Release, 2007, 122:63-70); Aliabadi et al. (International Journal of Pharmaceutics, 2007, 329:158-165); Aliabadi et al. (Journal of Pharmaceutical Sciences, 2008, 97(5):1916-1926); Binkhathlan et al. (European Journal of Pharmaceutics and Biopharmaceutics, 2010, 75:90-95); Aliabadi et al. (Journal of Controlled Release, 2005, 104:301-311), all herein incorporated by reference in their entirety. The micelles described in these references can be used to deliver one or more cardioactive agents in the treatment or prevention of heart failure.

In an example, a PEO-b-PCL micelle comprising PEO-b-PCL block copolymer exhibiting a molecular weight of greater than about 6000 Daltons can be used. In one aspect, the molecular weight of the copolymer is between about 10,000 and about 29,000 Daltons. In another aspect, the molecular weight of the copolymer is about 18,000 Daltons. In another aspect, the PEO molecular weight is about 5000 Daltons or greater. In a further aspect, the PCL molecular weight is about 5000 Daltons or greater. In another aspect, the micelle further comprises a biologically active agent. In another aspect, the agent is hydrophobic.

The micelle may be formed by a method, namely: a. obtaining a solution of amphiphilic block copolymers in a water miscible solvent; b. combining the solution of amphiphilic block copolymers with a suitable aqueous medium under conditions sufficient to minimize aggregation; and c. removing the water miscible organic solvent. The water-miscible solvent may be acetone, tetrahydrofuran (THF), dimethyl sulfoxide (DMSO), dimethyl formamide (DMF), dimethyl acetamide (DMAC), acetonitrile, or suitable mixtures thereof. The aqueous medium may be water, saline, 5% dextrose or isotonic sucrose. The ratio of the solution of amphiphilic block copolymers to aqueous medium may be between about 1:2 and about 1:10. In an aspect, the micelle further comprises adding the cardioactive agent in step (a). The micelle can have an average diameter up to about 500 nm, in the range of from about 50 nm to about 150 nm, in the range of from about 55 to about 100 nm, in the range of about 55 to about 125 nm, or more typically, in the range of about 100 to about 125 nm.

Although not wishing to be bound to any particular theory, the use of higher molecular weight PEO polymers (e.g. 114 monomers in the PEO block) in the structure of block copolymer may result in less aggregation of micelle particles and modified, i.e. enhanced, biodistribution, decreased toxicity, and improved therapeutic efficacy.

Also described herein are compositions comprising an amphiphilic block copolymer and a cardioactive agent, in which the amphiphilic block copolymer forms a micelle around the cardioactive agent. In a more particular embodiment of the invention, the amphiphilic block copolymer forms a micelle around the cardioactive agent by one or more of chemical conjugation, electrostatic complexation, and physical encapsulation.

The amphiphilic block copolymer micellar solutions may be prepared in isotonic medium and administered intravenously. The micelles may, therefore, be suitably formulated into pharmaceutical compositions for administration to human subjects in a biologically compatible form suitable for administration in vivo. Accordingly, in another aspect, the pharmaceutical composition comprises the micelles, in admixture with a suitable diluent or carrier. The compositions containing the micelles can be prepared by known methods for the preparation of pharmaceutically acceptable compositions which can be administered to subjects, such that an effective quantity of the cardioactive agent within the micelles is combined in a mixture with a pharmaceutically acceptable vehicle. Suitable vehicles are described, for example, in Remington's Pharmaceutical Sciences (2003-20th edition), in The United States Pharmacopeia: The National Formulary (USP 24 NF19) published in 1999 and in the Handbook of Pharmaceutical Additives (compiled by Michael and Irene Ash, Gower Publishing Limited, Aldershot, England (1995)).

On this basis, the compositions include solutions of the micelles in association with one or more pharmaceutically acceptable vehicles or diluents. The solutions are buffered to a suitable pH and iso-osmotic with physiological fluids. In this regard, reference can be made to U.S. Pat. No. 5,843,456, which is incorporated herein by reference. In one aspect, the pharmaceutical compositions can be used to enhance biodistribution and drug delivery of hydrophobic drugs. In accordance with the methods of the invention, the described micelles may be administered to a subject in a variety of forms depending on the selected route of administration, as will be understood by those skilled in the art. The micelles of the invention are intended to be administered parenterally, e.g. via intravenous, subcutaneous, intramuscular, transepithelial, intrapulmonary, and topical modes of administration. Parenteral administration may be by continuous infusion over a selected period of time. Preferably, the micelles are administered by injection subcutaneously or intravenously. Embodiments of the micelles are effective to enhance the permeability of drugs across the blood brain barrier. Solutions of a micelle can be prepared in water mixed with suitable excipients. Under ordinary conditions of storage and use, these preparations contain a preservative to prevent the growth of microorganisms.

A person skilled in the art would know how to prepare suitable formulations. The pharmaceutical forms suitable for injectable use include sterile aqueous solutions or dispersion and sterile powders for the extemporaneous preparation of sterile injectable solutions or dispersions. In all cases the form is sterile and must be fluid to the extent that easy syringability exists.

Compositions for nasal administration may conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. The aerosol dosage forms can also take the form of a pump-atomizer.

Compositions for rectal administration are conveniently in the form of suppositories containing a conventional suppository base such as cocoa butter.

In embodiments, a delivery system (e.g. an implantable device) can be used to deliver compositions according to the invention, wherein the composition comprises micelles carrying cardioactive agents, e.g. hydrophobic cardioactive agents. The hydrophobic cardioactive agents can be loaded into micelles comprising a hydrophobic core and a hydrophilic outer surface, thus improving the delivery of the hydrophobic cardioactive agents in aqueous mediums, such as blood and body fluids. On the other hand, hydrophilic cardioactive agents can be loaded into micelles via chemical conjugation to the hydrophobic core of the micelle with the hydrophilic outer surface of the micelle being used to facilitate delivery in aqueous mediums, such as blood and body fluids. The present micelles can help to reduce the toxicity profile of the cardioactive agent.

Methods of Treatment

Described herein are surprising findings that, in aspects, certain micelle(s) described herein (e.g. nanoparticles) can preferentially and passively accumulate or localize in fibrotic areas (in association with fibroblasts) of the heart of subjects suffering from heart failure, such as HFpEF. In aspects, the micelle(s), micelle-forming amphiphilic block copolymer(s), and/or composition(s) described herein (e.g. nanoparticles) can deliver effective amounts of therapeutics to such tissue in heart failure patients while mitigating or avoiding the risk of systemic toxicity.

Without being bound by theory, it is believed that the micelle(s) described herein (e.g. nanoparticles) can preferentially accumulate or localize in areas in which fibroblasts are present, including in fibroblasts themselves, using the enhanced permeability and retention (EPR) effect as a result of the size of the micelle(s) described herein (e.g. nanoparticles) and the disrupted endothelium and hyperpermeability of the inflamed vasculature in the locality of the fibrous tissue. In aspects, the heart failure milieu can be characterized by local inflammation, hypoxia, oxidative stress, impaired lymphatic drainage-conditions similar to those observed in the tumour micro-environment and in the peri-infarct zone of myocardial infarction. EPR results from inflammation and is independent of fibrosis. In aspects, the present micelles are delivered passively to fibroblasts in heart failure subjects in contrast to active targeting involving the use of ligands that bind to cell receptors. In other aspects, the present nanoparticles are not modified to bind to any cell receptors present in inflamed or fibrous areas of the heart. It is speculated that fibrous heart tissue has a more open structure than the surrounding non-fibrotic cardiomyocytes and the micelles become ‘caught’ in the fibrous extracellular matrix (ECM).

The present micelles can be used to deliver cardioactive agents to fibrotic areas of the heart to treat heart failure. Examples of such drugs include cannabidiol (CBD) which has been shown to reduce inflammation and fibrosis in an animal model of autoimmune myocarditis (Lee et al., Mol Med. 2016; 22: 136-146); methotrexate, which has been shown to reduce fibrosis in a rat model of autoimmune myocarditis (Zhang et al, Mediators of Inflammation Volume 2009, Article ID 389720); rapamycin, which has been shown to reduce cardiac fibrosis in an model of uremic cardiac fibrosis (Haller et al, J Am Heart Assoc. 2016 October; 5(10): e004106); and thalidomide, which has been shown to reduce aspects of cardiac fibrosis in a post myocardial infarct animal model (Yndestad et al, European Journal of Heart Failure. 2006; 8: 790-796).

Heart failure, often referred to as congestive or chronic heart failure (CHF), occurs when the heart is unable to pump sufficiently to maintain blood flow to meet the body's needs. Signs and symptoms of heart failure commonly include shortness of breath, excessive tiredness, and leg swelling. The shortness of breath is usually worse with exercise, while lying down, and may wake the person at night. A limited ability to exercise is also a common feature. Chest pain, including angina, does not typically occur due to heart failure. Heart failure is a common, costly, and potentially fatal condition. In 2015 it affected about 40 million people globally. Overall around 2% of adults have heart failure and in those over the age of 65, this increases to 6-10%. This is a serious disease with significant morbidity and a high mortality; the 5 year survival for symptomatic heart failure is only approximately 50% and therefore worse than many cancers.

Common causes of heart failure include coronary artery disease including a previous myocardial infarction (heart attack), high blood pressure, atrial fibrillation, valvular heart disease, excess alcohol use, infection, and cardiomyopathy of known (e.g. diabetic cardiomyopathy) or unknown cause. These cause heart failure by changing either the structure or the functioning of the heart. Heart failure is not the same as myocardial infarction (in which part of the heart muscle dies) or cardiac arrest (in which blood flow stops altogether).

There are several terms which are closely related to heart failure and may be the cause of heart failure but should not be confused with it. Cardiac arrest and asystole refer to situations in which there is no effective contraction of the heart and therefore no cardiac output at all. Without urgent treatment, these result in sudden death. Myocardial infarction (“heart attack”) refers to heart muscle damage due to insufficient blood supply, usually as a result of a blocked coronary artery. Cardiomyopathy refers specifically to problems within the heart muscle, and these problems can result in heart failure. Ischemic cardiomyopathy implies that the cause of muscle damage is coronary artery disease. Dilated cardiomyopathy implies that the muscle damage has resulted in enlargement of the heart. Hypertrophic cardiomyopathy involves enlargement and thickening of the heart muscle.

There are many different ways to categorize heart failure, including:
the side of the heart involved (left heart failure versus right heart failure). Right heart failure compromises pulmonary arterial flow to the lungs. Left heart failure compromises aortic flow to the body and brain. Mixed presentations are common; left heart failure often leads to right heart failure in the longer term.
whether the abnormality is due to insufficient contraction (systolic dysfunction), or due to insufficient relaxation of the heart (diastolic dysfunction), or to both.
whether the problem is primarily increased venous back pressure (preload), or failure to supply adequate arterial perfusion (afterload).
whether the abnormality is due to low cardiac output with high systemic vascular resistance or high cardiac output with low vascular resistance (low-output heart failure vs. high-output heart failure).
the degree of functional impairment conferred by the abnormality (as reflected in the New York Heart Association Functional Classification)
the degree of coexisting illness: i.e. heart failure/systemic hypertension, heart failure/pulmonary hypertension, heart failure/diabetes, heart failure/kidney failure, etc.

Functional classification generally relies on the New York Heart Association functional classification. The classes (I-IV) are:

Class I: no limitation is experienced in any activities; there are no symptoms from ordinary activities.

Class II: slight, mild limitation of activity; the patient is comfortable at rest or with mild exertion.

Class III: marked limitation of any activity; the patient is comfortable only at rest.

Class IV: any physical activity brings on discomfort and symptoms occur at rest.

This score documents the severity of symptoms and can be used to assess response to treatment. While its use is widespread, the NYHA score is not very reproducible and does not reliably predict the walking distance or exercise tolerance on formal testing.

In its 2001, guidelines the American College of Cardiology (ACC)/American Heart Association working group introduced four stages of heart failure: Stages A, B, C, and D. Stage A refers to patients at high risk for developing HF in the future but who have no functional or structural heart disorder. Stage B refers to patient having a structural heart disorder but no symptoms at any stage. Stage C refers to patients with previous or current symptoms of heart failure in the context of an underlying structural heart problem which symptoms are managed with medical treatment. Stage D refers to patients having advanced disease and requiring hospital-based support, a heart transplant or palliative care.

The ACC staging system is useful in that Stage A encompasses “pre-heart failure”—a stage where intervention with treatment can presumably prevent progression to overt symptoms. ACC Stage A does not have a corresponding NYHA class. ACC Stage B would correspond to NYHA Class I. ACC Stage C corresponds to NYHA Class II and III, while ACC Stage D overlaps with NYHA Class IV.

Heart failure represents a leading cause of death and disability with associated U.S. health care costs exceeding $30 billion annually. Over 5 million adults in the U.S. suffer from heart failure with a 5 year mortality of 50%. 50% of all heart failure patients have heart failure with preserved ejection fraction (HFpEF). There have been no significant treatment advances in HFpEF in over 20 years. The main therapy involves the use of diuretics.

Whatever the cause of CHF, its development is associated with significant inflammation within the heart tissue and vasculature, enlargement of cardiomyocytes (hypertrophy), and with a significant increase in fibrous tissue rendering the myocardium stiff, thereby restricting cardiac filling and output. Inflammation in the cardiac tissue is associated with an increased number of inflammatory cells, increased levels of inflammatory cytokines, increased numbers of fibroblasts and fibrous tissue, decreased contractile function of the myocytes, increased oxidative stress and increased cell death. See Inflammation in Heart Failure. Circ Res. 2015; 116: 1254-1268 (the “Mann paper”) which reviewed the role of immune responses in the heart and describes negative outcomes of clinical trials designed to address inflammation in heart failure. The Mann papers teaches that inflammatory cytokines provoke left ventricular dysfunction and hence reduced blood flow in the circulation, which is a key aspect of heart failure. It also teaches that inflammatory mediators are involved in LV ventricular remodelling, which are changes that occur in cardiac shape, size, and composition in response to myocardial injury. These changes include cardiac myocyte hypertrophy, myocardial fibrosis, as well as progressive myocyte loss through apoptosis.

Although there is increasing evidence of the involvement of inflammation in heart failure, the use of anti-inflammatory approaches such as those used successfully in rheumatoid arthritis (Enbrel etc.) have not only failed in HF but in some cases have exacerbated the condition, and, for at least this reason, the success of any anti-inflammatory therapy in heart failure such as that described herein is considered surprising.

Inflammatory cytokines have also been associated with arrhythmias arising in post myocardial infarction (MI) situations. See Stuart et al, 2016: Journal of Molecular & Cellular Cardiology 91: 114-122. This review paper describes various pathways by which both inflammatory cytokines and fibrosis may facilitate arrhythmias. The inventors believe therefore that the present technology which is useful in delivering cardioactive agents such as anti-inflammatory and antifibrotic agents to the heart would be useful in treating inflammation and pathologies linked to inflammation such as heart failure and cardiac arrhythmia.

The present micelles, and compositions and drug delivery systems containing same, are non-toxic, provide an increase in control of drug release and improved biodistribution as the micelles can be designed so as not to aggregate in the composition. In some embodiments, the micelles carry hydrophobic biologically active agents and are formed from self-assembly of the amphiphilic block copolymers. In an embodiment, the micelles are composed of copolymers of high molecular weight. In another embodiment, the PEO-b-PCL copolymer used in micelle formation exhibits a molecular weight of greater than 6000 Daltons and in another embodiment exhibits a molecular weight of greater than 10000 Daltons. In one embodiment the micelles are formed using copolymers of molecular weight of about 7000-29000 Daltons. In another embodiment, formation of the micelles involves the use of a water miscible solvent. In one embodiment, the resultant micelles have an average diameter of less than 100 nm in the absence of agent, suitably 55-90 nm, or 20-60 nm in size. In another embodiment, agent loaded micelles have an average diameter of less than 200 nm, suitably 60-125 nm in size. In one embodiment, more than one type of biologically active agent is loaded into the micelle. Parameters of micellar drug delivery can be modified by modifying particle size, while having sufficient amount of drug loaded in the micelle for drug delivery.

The micelles, and compositions and drug delivery vehicles containing same, can be used to target fibroblasts and thereby treat or prevent heart failure.

The micelles can be administered to an animal alone or in combination with pharmaceutically acceptable carriers, as noted, the proportion of which is determined by the solubility and chemical nature of the composition, chosen route of administration and standard pharmaceutical practice. In an embodiment, the pharmaceutical compositions are administered in a convenient manner such as by direct application to the affected site, e.g. by injection (subcutaneous, intravenous, etc.) or by diffusion or release from an implantable device. Typically, the composition is administered systemically. It may be desirable to administer the micelles of the invention and compositions comprising same, through known techniques in the art, for example by inhalation. Depending on the route of administration (e.g. injection, oral or inhalation, etc.), the pharmaceutical compositions or micelles or cardioactive agents in the micelles of the invention may be coated in a material to protect the micelles or agents from the action of enzymes, acids and other natural conditions that may inactivate the compound. In addition to pharmaceutical compositions, compositions for non-pharmaceutical purposes are also included within the scope of the present invention, such as for diagnostic or research tools. In one embodiment, the cardioactive agents or micelles comprising said drugs can be labeled with labels known in the art, such as fluorescent or radio-labels or the like.

Another aspect of the invention includes a method of delivering cardioactive agents to treat heart failure or cardiac arrhythmia in a subject in need thereof comprising administering an effective amount of an agent-loaded micelle of the invention to said subject. The dosage of the micelles of the invention can vary depending on many factors such as the pharmacodynamic properties of the micelle, the cardioactive agent, the rate of release of the agent from the micelles, the mode of administration, the age, health and weight of the recipient, the nature and extent of the symptoms, the frequency of the treatment and the type of concurrent treatment, if any, and the clearance rate of the agent and/or micelle in the subject to be treated. One of skill in the art can determine the appropriate dosage based on the above factors. The micelles may be administered initially in a suitable dosage that may be adjusted as required, depending on the clinical response. For ex vivo treatment of cells over a short period, for example for 30 minutes to 1 hour or longer, higher doses of micelles may be used than for long term in vivo therapy. The micelles can be used alone or in combination with other agents that treat the same and/or another condition, disease or disorder. In another embodiment, where either or both the micelle or cardioactive agent is labeled, one can conduct in vivo or in vitro studies for determining optimal dose ranges, drug loading concentrations and size of micelles and targeted drug delivery for a variety of diseases.

For example, in some aspects, the micelles described herein and/or the cardioactive agent within the micelles described herein may be administered in an amount of from about 0.001, 0.01, 0.1, 1, 10, 15, 20, 25, 50, 75, 100, 125, 150, or 175 mg/kg body weight, and/or up to about 1000, 900, 800, 750, 700, 600, 500, 450, 400, 350, 300, 250, 200 mg/kg body weight, per week, per day, per hour, or per dose. All intermediate values and permutations and combinations of these values are also intended to be covered.

Furthermore, the cardioactive agent may be used in any ratio with the amphiphilic block copolymer, such as from about 0.05 to about 0.4, typically, about 0.1 to about 0.3. The weight ratio can be any suitable ratio and typically, the ratio is such that the micelle formed remains within a suitable particle size as described herein.

It will be understood that the micelles described herein comprising a cardioactive agent may be administered in combination with a conventional cardioactive agent, which may be the same or different from the cardioactive agent comprised within the micelles. The combination may be administered concurrently or consecutively, in any order. In aspects, the micelle-bound cardioactive agent and the conventional cardioactive agent act additively or synergistically to treat and/or prevent heart failure in a subject.

It will also be understood that, by shielding or sequestering a cardioactive agent in the micelles described herein, the cardioactive agent will have fewer systemic effects than what might be observed if the cardioactive agent was administered without the micelle. This allows for more targeted activity of the cardioactive agent, as it will release and accumulate in the desired tissue.

Without wishing to be bound by theory, it is believed that the micelles described herein localize to cardiac fibroblasts through a size-based mechanism. The initial stage of fibrosis in the heart is interstitial fibrosis and involves a dense perivascular network of collagen extracellular matrix laid down by myofibroblasts. Based on the findings described herein, it is believed that enhanced permeation in the vascular network of the heart will result in the micelles accumulating initially in this area of fibrosis. It is believed that micelles of a size up to about 500 nm, and more typically in the range of from about 50 nm to about 150 nm, become entrapped in the fibrous extra cellular matrix and accumulate in fibroblasts in a size dependent manner.

Also described herein is a method of delivering a cardioactive agent to a subject, comprising administering to the subject an amphiphilic block copolymer which is capable of forming a micelle around an effective amount of the cardioactive agent.

As shown herein, it has been found that cardioactive agent-containing micelles formed from amphiphilic block copolymers passively accumulate in heart tissue and preferentially localize to cardiac fibroblasts as compared to non-fibrotic areas of the heart. For example, in some aspects, the ratio of localization in or around fibrotic tissue as compared to non-fibrotic tissue is from about 10,000:1 to about 2:1, such as from about 10,000, about 5,000, about 2,500, about 2,000, about 1,500, about 1,000, about 900, about 800, about 700, about 600, about 500, about 400, about 300, about 200, about 150, about 100, about 90, about 80, about 70, about 60, about 50, about 40, about 30, about 20, about 10, about 9, about 8, about 7, about 6, about 5, about 4, about 3, or about 2 to 1.

Typical cardioactive agents for use with the micelles described herein are for treating and/or preventing fibrosis and/or inflammation, such as neuroinflammation. These include methotrexate, cannabidiol, cyclosporine A, derivatives thereof, and combinations thereof. Without wishing to be bound by theory, it is believed that cyclosporine A (CsA) may be useful in preventing cardiomyocyte death based on the following: the proton gradient across the mitochondrial membrane is lost as mitochondria become depolarized, leading to cell death. The mitochondrial permeability transition pore (MPTP) is important in this process, and loss of cyclophilin D from the MPTP protects against cardiomyocyte death (Di Lisa et al. Biochim Biophys Acta 2011; 1813: 1316-1322). CsA binds to cyclophilin D, inhibiting MPTP opening and preventing cardiomyocyte cell death (Hausenloy et al. Br. J. Pharmacol. 2012; 165: 1235-1245).

CsA attenuates myocardial injury in in vivo models of ischaemia-reperfusion injury (Argaud et al. J Mol Cell Cardiol. 2005; 38:367-74). A pilot study of patients with acute ST-elevation MI indicated that administration of CsA at the time of percutaneous coronary intervention limits reperfusion injury and reduces infarct size (Piot et al N Engl J Med 2008; 359:473-481). However, the results of the CIRCUS trial failed to confirm this—there were no significant differences in serious cardiovascular events between the CsA and placebo groups (Cung et al. N Engl J Med. 2015; 373:1021-31).

There is evidence from transgenic HIF1-a mice that overexpress HIF1-a that this protein protects the heart from an acute ischaemic insult. This may be attributed to an increased capillary area in the heart and metabolic preconditioning via increasing gene expression and glucose metabolism necessary for the anaerobic metabolic switch. They also show altered Ca⁺⁺ handling. However, these same pathways are indicative of defective cardiac homeostasis in the failing heart and, as these HIF1-a overexpressing mice age, they develop heart failure. Furthermore, increased levels of HIF1-a levels have been found in patients with dilated cardiomyopathy, indicating a chronic activation of the HIF pathway in these heart failure patients (Holscher et al. Cardiovascular Research, 2012; 94: 77-86).

In a model of fibrotic lung injury induced by bleomycin and involving the upregulation of TGF-beta, HIF1-a was shown to be involved in upregulation of fibrosis. CsA significantly reduced the expression of HIF1-a and fibrosis. In vitro, CsA inhibited TGF-beta-induced myofibroblast formation by enhancing protein degradation of HIF-1a. Similarly, CsA and inhibition of HIF-1a dedifferentiated myofibroblast-like cells that were derived from a patient with pulmonary fibrosis (Yamazaki et al. FASEB J., 2017; 31, 3359-3371).

The micelle, micelle-forming amphiphilic block copolymer, composition and/or the drug delivery device described herein may be used to attenuate cardiac dysfunction, decrease oxidative stress, fibrosis and/or inflammation; avoid first pass metabolism; reduce requirement for high dose therapy; reduce toxicity of an agent; improve safety profile; support sustained drug release; and/or improve bioavailability and the pharmacokinetic profile.

The above disclosure generally describes the present invention. A more complete understanding can be obtained by reference to the following specific examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples of embodiments of the invention are intended to be illustrative and not to limit the remainder of the disclosure.

### EXAMPLES

### Example 1: Synthesis of Cy5.5 conjugated poly(ethylene oxide)-block-poly(a-benzyl carboxylate-e-caprolactone) (PEO-PBCL)

PEO-PBCL was synthesized by ring-opening polymerization α-benzyl carboxylate-ε-caprolactone (0.2 g), using methoxy-PEO (MW: 5000 g/mol) (0.5 g) as initiator and stannous octoate as catalyst according to a method described previously (Mahmud et al, Macromolecules, 2006). Prepared PEO-PBCL were end capped with α-propargyl carboxylate-ε-caprolactone (PCC) using stannous octoate as catalyst. Briefly, PEO-PBCL (0.2 g) and PCC (0.021 g) were added to a 25 mL round-bottom flask previously filled with κ mL dry toluene under constant stirring. Stannous octoate (0.010 equivalent of monomer) was added to the flask. The flask was then refluxed for 30 h. The reaction was terminated by cooling the product to room temperature. The product was then precipitated in hexane and the supernatant was discarded. The final product was washed with ether and dried under vacuum for further use.

Near-infrared fluorophore (NIRF) Cy5.5-azide was conjugated to the terminal alkyne of PCC in PEO-PBCL-PCC using Huisgens 1,3-dipolar cycloaddition (azide-alkyne click chemistry) reaction. The terminal alkyne group of PCC reacted with the terminal azide group of Cy5.5 azide to form a 1,3-triazole ring. Cu(I) acts as a catalyst for the reaction. Cu(I) is prepared in situ by the addition of Cu(II) TBTA Complex, and ascorbic acid, reducing Cu(II) to Cu(I). Briefly, PEO-PBCL-PCC (112 mg) was dissolved under constant stirring in a 10 mL round-bottom flask containing 2 mL degassed DMSO. Cy5.5 azide (1 μmol; 0.7 mg) was dissolved in 400 μL DMSO and added to the mixture under constant stirring followed by addition of ascorbic acid (0.5 μmol; 0.1 mg) previously dissolved in 100 μL water. The flask was then degassed with argon for about 30 s. 10 mM Cu-TBTA Complex solution (0.5 μmol; 60 μL) was finally added followed by degassing for 30 s using argon. The reaction mixture was sealed and incubated with stirring at room temperature in the dark for 16 h. Argon was flushed through the sealed vial at 4 and 8 h time-points. After incubation, the mixture was separated from the non-reacted dye by dialysis against DMSO for 24 h followed by dialysis against water for 24 h to remove the DMSO, and then lyophilized. Prepared block copolymers were characterized by ¹H NMR.

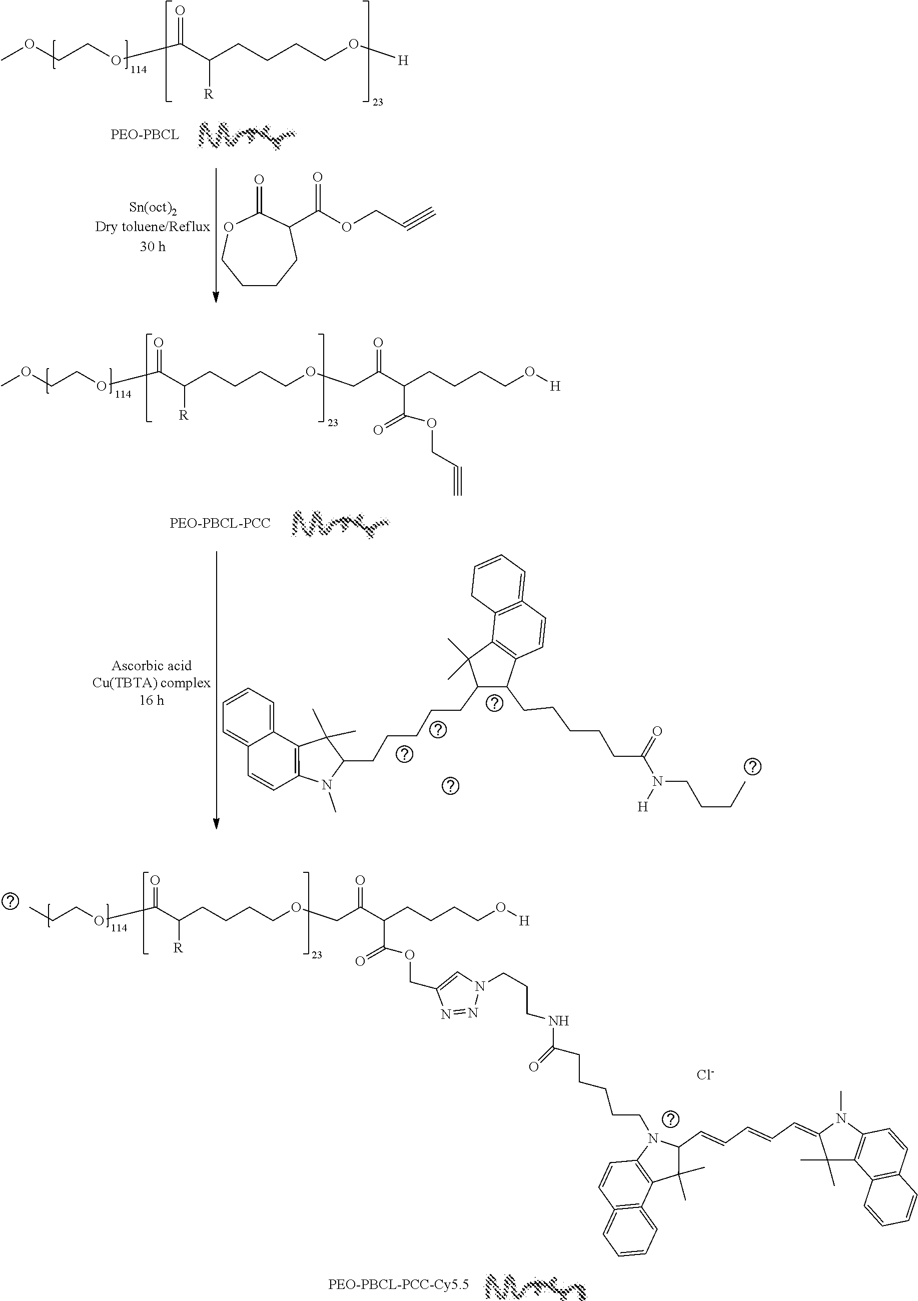

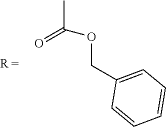

### Example 2: Preparation of Cy5.5 Labelled Micelles

Cy5.5-labeled block copolymer micelles were prepared by co-solvent evaporation method. Briefly, PEO114-PBCL23 (18.88 mg) and PEO-PBCL-PCC-Cy5.5 (1.12 mg) were mixed and dissolved in acetone (0.4 mL). The solution was added to 4 mL of doubly distilled water in a drop-wise manner under moderate stirring at room temperature, followed by evaporation of acetone under vacuum. The prepared micellar solution was then centrifuged to remove any aggregates.

The polymers and micelles had the following characteristics:
Degree of polymerization for PEO=114 as defined by manufacturer and for PBCL=23 as defined by ¹H NMR.
Average Micelles diameter (Z Average)=52.94 nm (This is for the equivalent micelles without dye as presence of Cy5.5. makes the polymer incompatible with laser beam used in DLS instrument used to measure micellar hydrodynamic size.
Polydispersity index=0.349

### Example 3: In Vivo Studies of Fluorescently-Labelled PEO-PBCL Nanoparticles

Fluorescently-labelled PEO-PBCL nanoparticles were administered by injection to an animal model of heart failure based on the model described by Oestreicher et al, 2003 (Circulation. 2003; 108:2517-2523). The animal model consisted of 1 week ad libitum administration of water containing 1% NaCl and 0.01% of N-nitro L-arginine methyl ester (l-NAME), then a micro-osmotic pump was surgically implanted in the subdermal dorsal area, infusing angiotensin II at a rate of 0.7 mg/kg/day over a course of 28 days. By the fifth week heart failure was present and the nanoparticles were injected. This model demonstrates parameters of cardiac dysfunction, such as the increase of hormones associated with heart failure (elevation of BNP), changes in inflammatory markers, adverse remodelling, depression in ejection fraction and cardiomyopathy, characteristic of heart failure.

Fluorescently-labelled PEO-PBCL nanoparticles were shown to accumulate in the heart in this model of heart failure following IV, SC, and IP administration compared to control animals injected with the fluorescent nanoparticles (FIG. 1; Note, the failing hearts (HF) were enlarged compared to the control hearts and showed enhanced fluorescence indicating accumulation of the fluorescently labelled nanoparticles in the failing hearts).

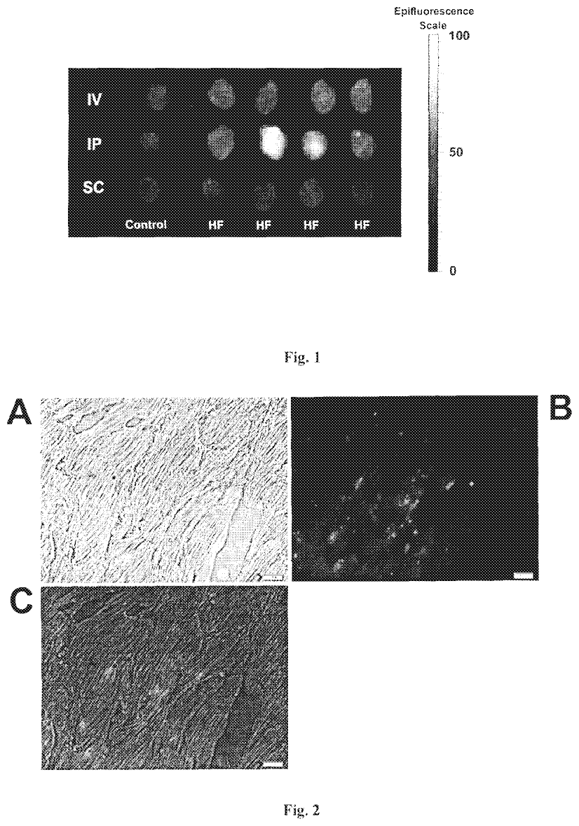

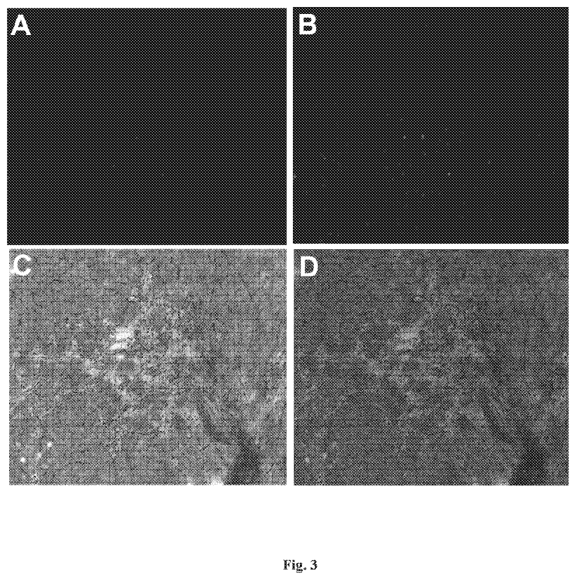

Overall, fluorescence was greater in heart failure cardiac tissue than in control cardiac tissue. As shown in FIG. 2, uptake of fluorescently-labelled nanoparticles administered by SC injection was demonstrated in cardiac tissue of heart failure mice and there were localized deposits of nanoparticle accumulations associated with some cells.

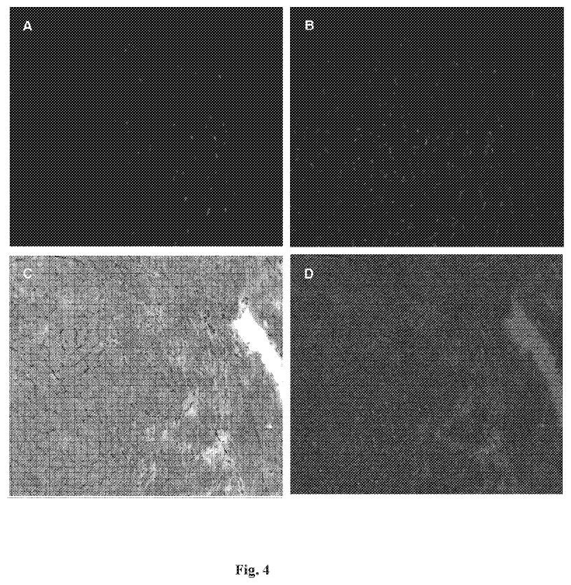

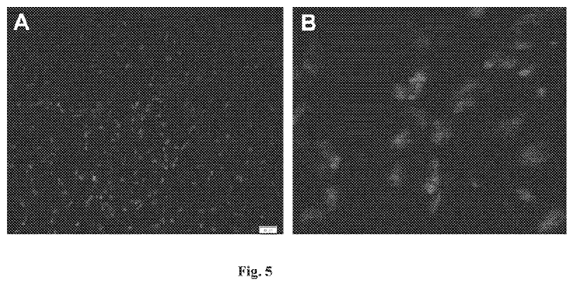

Higher concentrations of nanoparticles were associated with spindle-shaped non-contractile cells within areas of fibrous tissue rather than with contractile cells, as shown in FIGS. 3 and 4. These highly fluorescent areas with the highest accumulation of nanoparticles were not seen in control cardiac tissues and were only found in smaller non-contractile cells. This area of fibrous tissue was clearly seen in the H&E staining.

The non-contractile cells with the highest accumulation of nanoparticles are primarily spindle shaped and associated with the areas of fibrous tissue and are most likely fibroblasts. In conclusion, the principal accumulation of nanoparticles was seen associated with fibroblasts within areas of fibrous tissue in the heart and not endothelial cells or contractile myocytes.

### Example 4: Synthesis of a Methotrexate-Carrying Micelle

A micelle carrying methotrexate (R₁) attached, via a spacer (L₁), to an amphiphilic block copolymer, PEG-PCCL, was prepared. The PEG-PCCL to which methotrexate is covalently bound to the PCCL portion is shown below:

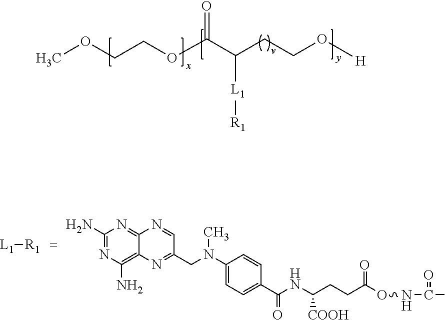

Wherein L₁ is O—(CH2)n-NH—, and wherein n is 2-6. Examples of L₁ include 2-aminoethanol and 6-amino hexanol. x, v, y are as described herein.

The micelle was prepared by dissolving 0.8 g of polymer (PCCL or PEG-PCCL) in 5 mL of anhydrous dimethyl formamide (DMF). The carboxylic acid functional groups were activated to react to the amine group of the spacer (L₁) (which in this case was 2-aminoethanol). Carboxylic acids activate with DCC/NHS (N,N′-dicyclohexylcarbodiimide/N-hydroxysuccinimide) or similar compounds or chlorination. An equivalent amount of 2-aminoethanol was added to the solution containing activated polymer in DMF. The reaction container was stirred and left overnight at room temperature. The reaction mixture was purified with dialysis against dimethyl sulfoxide (DMSO) and water. The solution was freeze dried to produce the PEG-PCCL polymer with the spacer L₁.

An amount of methotrexate (MTX) was activated with carbodimide compounds such as DCC/DMAP (4-Dimethylaminopyridine) in anhydrous DMF. The above-mentioned product was dissolved in anhydrous DMF. The polymer solution was added to the activated MTX. The reaction mixture was stirred and left at room temperature overnight. The reaction mixture was purified by dialysis against DMSO and then water. The solution was then freeze dried.

### Example 5: Preparation of a Cyclosporine A-Carrying Micelle

Poly(ethylene oxide)-poly(ε-caprolactone) (PEO:PCL) block co-polymer was synthesized and used to prepare a micellar solution in which cyclosporine A was encapsulated in nano-sized micelles (also referred to herein as nanoencapsulated CsA) in accordance with the procedures described by H. M. Aliabadi et al. Biomaterials 26 (2005) 7251-7259, the contents of which are incorporated herein by reference.

### Example 6: In Vivo Studies Using Nanoencapsulated Cyclosporine A

A study was conducted to investigate the efficacy of free cyclosporine A (CsA) versus the nanoencapsulated CsA prepared in Example 5 in a murine model of non-ischemic heart failure (HF).

Method

Heart failure was induced in mice in accordance with the animal model of heart failure described by Oestreicher et al, 2003 (Circulation. 2003; 108:2517-2523) incorporated herein by reference. Briefly, water containing 1% NaCl and 0.01% N-nitro L-arginine methyl ester (1-NAME) was administered to mice for 7 days, followed by surgical implantation of micro-osmotic pumps to infuse angiotensin II at 0.7 mg/kg/day over 4 weeks. At 5 weeks, HF had been induced as demonstrated by parameters of cardiac dysfunction such as increased levels of hormones associated with heart failure and changes in inflammatory markers.

Four groups of animals were used, two test groups and two control groups. A first test group comprising three mice in which heart failure was induced received CsA (ANG+CsA). A second test group comprising five mice received nanoencapsulated CsA (ANG+NCsA). A first control group comprised three mice in which HF was induced (ANG) and a second control group comprised three mice with healthy hearts (CTRL), i.e. in which heart failure was not induced. The drugs were administered to the test groups by subcutaneous injection at a dose of 1 mg/kg/every third day following induction of HF for four weeks. The mice in all groups were sacrificed and their heart tissues extracted, frozen, and sections taken for fluorescent microscopic examination.

Results

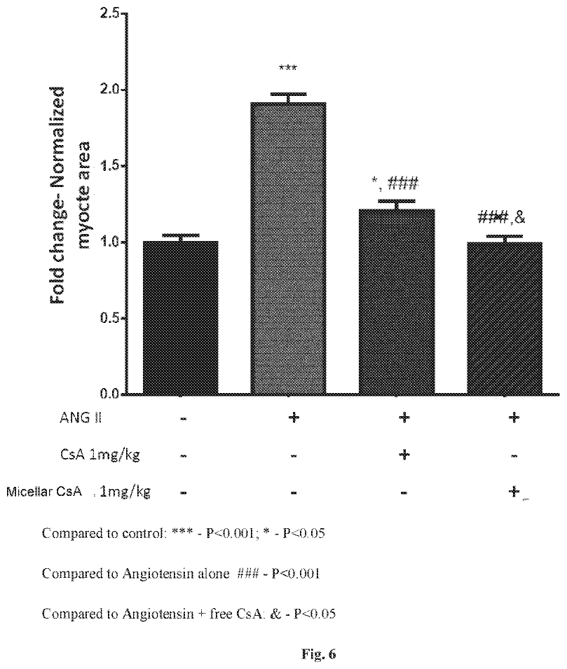

Histological examination showed an increase in enlargement of cardiomyocytes and a concomitant increase in fibrosis in mice in which HF was induced (ANG II), compared with healthy hearts (CTRL). The normalized diameters of cardiomyocytes from the control and treated hearts are depicted in FIG. 6. These results show the nanoencapsulated CsA (also referred to herein as micellar CsA) is superior to free CsA in reducing cardiomyocyte size in a murine model of non-ischemic heart failure.

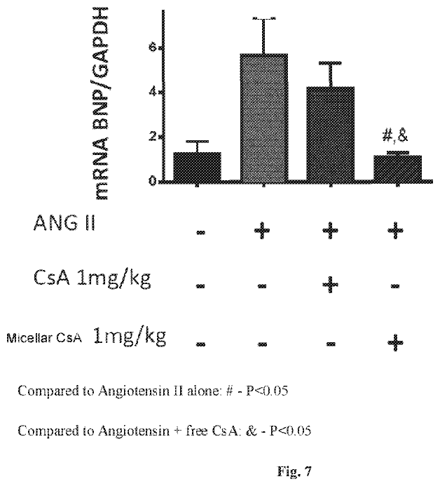

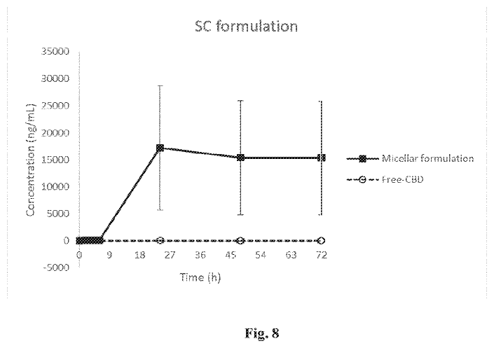

The remodeling tissue marker, B-type Natriuretic Peptide (BNP), is secreted by cardiac fibroblasts, and circulating levels of BNP correlate with left ventricular fibrotic mass (cardiac fibrosis—Miyaji et al., 2016, Internal Medicine: 55; 1261-1268) in patients with hypertrophic cardiomyopathy and heart failure. BNP is elevated in heart failure induced mice and levels of BNP is correlated with severity of heart failure. Measurements of the cardiac mRNA expression of BNP following subcutaneous administration of free CsA versus micellar CsA are shown in FIG. 7, from which it can be seen that there is an increase in BNP in mice in which heart failure was induced (ANG II) as compared with that of healthy mice, and this is not significantly reduced by free CsA but is significantly reduced by micellar CsA. This is evidence that fibrosis was somewhat reduced in animals treated with free CsA (ANG II—CsA) and was significantly and greatly reduced in animals treated with nanoencapsulated CsA(ANG II+NCsA).

These tests show micellar CsA as being superior to free CsA in reducing fibrosis and mRNA expression of BNP. The data supports the theory of the present invention that the block copolymers described herein can be effective in carrying therapeutic agents to fibrotic tissues in the heart or to sites of inflammation in the heart. It is expected that lower doses of drugs can be administered using the block copolymers described herein thereby allowing for reduced dosing regimens, and better targeting of the heart tissue with a consequent reduction in system toxicity.

### Example 7—Evaluation of Pharmacokinetic Parameters of Free Versus Encapsulated CBD

A study was performed in healthy rats to evaluate the pharmacokinetic parameters of free CBD versus CBD encapsulated in a block copolymer. The materials used in the study are summarized in Table 1 below.

TABLE 1

| Material | Characteristics | Provider | CAS number |
|---|---|---|---|
| Poly(ethylene glycol) | molecular weight | Sigma-Aldrich | 9004-74-4 |
| methyl ether | (MW) of 5000 Da, | (St. Louis, MO, USA) | |
| (also referred to as methoxy | corresponding to an | | |
| polyethylene oxide | average degree of | | |
| or methoxy PEO) | polymerization (DP) of 114. | | |
| α-benzyl carboxylate | molecular weight (MW) | Synthesized by | 268728-41-2 |
| ε-caprolactone (BCL) | of 5000 Da, corresponding | Alberta Research | |
| | to an average degree of | Chemicals, Inc. | |
| | polymerization (DP) of 114. | (Edmonton, AB, Canada). | |
| Cannabidiol (CBD) | oil | Dalton Pharma Services | 13956-29-1 |
| | | (Toronto, ON, Canada) | |
| Sesame oil | oil | Sigma-Aldrich (Now Millipore | 8008-74-0 |
| | | Sigma Canada Co. of | |
| | | Oakville, Ontario, Canada) | |
| Cannabidiol-D₃ solution | 100 μg/mL in | Sigma-Aldrich | |
| standard for LC-MS | methanol solution | (St. Louis, MO, USA) | |
| CBD standards for LC-MS | 1,000 μg/mL in P&T | Sigma-Aldrich | 13-956-29-1 |
| | methanol, 1 mL/ampule | (St. Louis, MO, USA) | |
| Cannabinol solution | 1,000 μg/mL in P&T | Sigma-Aldrich | 521-35-7 |
| for LC-MS (CBN) | methanol, 1 mL/ampule | (St. Louis, MO, USA) | |
| HPLC water | Solvent | Sigma-Aldrich | |
| | (clear liquid) | (St. Louis, MO, USA) | |
| n-Hexane | Solvent | Sigma-Aldrich | |
| | (clear liquid) | (St. Louis, MO, USA) | |
| Acetonitrile | Solvent | Sigma-Aldrich | |
| | (clear liquid) | (St. Louis, MO, USA) | |

Synthesis of Block Copolymer

Two different block copolymers, methoxy poly(ethylene oxide)-block-poly(α-benzyl carboxylate-ε-caprolactone) (PEO-b-PBCL), were synthesized by ring-opening polymerization by placing the following ingredients in Table 2 in an ampule which was then vacuum sealed and placed in a 140° C. oven for 4 hours.

TABLE 2

| Material | PEO₁₁₄-b-PBCL₁₅ | | PEO₁₁₄-b-PBCL₂₃ | |
|---|---|---|---|---|
| α-benzyl carboxylate | 0.4 | g | 0.6 | g |
| ε-caprolactone (BCL) | | | | |
| Poly(ethylene glycol) | 0.5 | g | 0.5 | g |
| methyl ether (also referred to | | | | |
| as methoxy polyethylene | | | | |
| oxide or methoxy PEO) | | | | |
| stannous octoate catalyst | 25-50 | μL | 25-50 | μL |

The ampule was stirred occasionally to observe the viscosity of sample. After the reaction was completed, the ampule was brought to room temperature. Polymer purification was done by washing the product using dichloromethane, hexane, and ether. The mass of the polymer was calculated to determine the yield. The polymer structure was confirmed, and degree of polymerization (DP) of the hydrophobic block was determined by proton nuclear magnetic resonance (¹H NMR) spectra based on previously published methods (Gang et al, Colloids & Surfaces B, 2015, 132:161-70, Mahmud et al., 2006, Macromolecules 39 (26), 9419-9428, and also U.S. Publication 20100069295 to Lavasanifar et al., the described methods of which are incorporated herein by reference). The DP of synthesized PEO-b-PBCL polymer (PEO₁₁₄-b-PBCLₓ) were 15 and 23. These block copolymers are also referred to herein as PEO₁₁₄-b-PBCL₁₅ and PEO₁₁₄-b-PBCL₂₃, respectively.

Preparation of Micellar Formulations Containing CBD and Using the Block Copolymer (PEO₁₁₄-b-PBCLₓ)

Micellar formulations comprising CBD oil from Dalton Pharma Services encapsulated in one of PEO₁₁₄-b-PBCL₁₅ and PEO₁₁₄-b-PBCL₂₃ block copolymers were prepared as follows.

100 mg of PEO-b-PBCL polymer (PEO₁₁₄-b-PBCLₓ) was dissolved in acetone (500 μL) until no precipitate remained. To this was added a solution comprising 10 mg CBD solubilized in acetonitrile. The resulting solution was vortexed and added dropwise to double-distilled water (ddH2O, 4 mL) while stirring. The solution was left to stir overnight to allow the acetone and acetonitrile to be evaporated and micelles to be formed (the “co-solvent evaporation method”). The next day, the resulting micellar solution was centrifuged and then the supernatant was separated. The free CBD was then removed from the micellar solution using a 0.22 μm filter syringe.

Characterization of Micellar Formulations

The above micellar formulations were characterized in terms of their micelle size, polydispersity index (PDI), Drug loading (DL %), and encapsulation efficiency (EE %), Micelle size and PDI were determined using a Zetasizer Nano ZS from Malvern Instruments (Montreal, QC, Canada). Drug loading (DL %), and encapsulation efficiency (EE %) were determined based on the below equations:

Drug loading (DL %)=(amount of encapsulated drug (mg)/amount of polymer used (mg))×100

Encapsulation efficiency (EE %)=(amount of encapsulated drug (mg)/total amount of drug used (mg))×100.

High-performance liquid chromatography (HPLC) was used to measure the amount of CBD encapsulated in micelles to use in the above equations. The HPLC was done using a Shimadzu LC-10AD HPLC System with a Shimadzu SPD-10A detector at 205 nm, and a Supelco Analytical Discovery C18 column (25 cm×4.6 mm, 5 μm). A mobile phase of 85:15 acetonitrile:water was used at a flow rate of 1 mL/min. and was delivered isocratically. The retention time was 5.75 mins.

The characteristics of the micellar formulations are summarized in Table 3 below.

TABLE 3
Characteristics of Micellar Formulations comprising
CBD encapsulated in PEO₁₁₄-b-PBCL₁₅
and PEO₁₁₄-b-PBCL₂₃ block copolymers

| | Polymer: | Micelle | | | |
|---|---|---|---|---|---|
| | CBD ratio | diameter | | | |
| Formulation | (W/W) | (nm) | PDI | DL % | EE % |
| Micellar CBD | 10:1 | 40.93 | 0.279 | 4.2 | 42 |
| (DP 23) | | | | | |
| Micellar CBD | 10:1 | 51.51 | 0.409 | 12.3 | 77 |
| (DP 15) | | | | | |

The data shows both PEO₁₁₄-b-PBCL₁₅ and PEO₁₁₄-b-PBCL₂₃ block copolymers are effective to form micelles having <100 nm average diameter. This suggests that these block copolymers will be effective to deliver CBD to fibrotic cells of the heart in a subject suffering from hear failure. The drug loading and encapsulation efficiency for PEO₁₁₄-b-PBCL₁₅ is higher than for PEO₁₁₄-b-PBCL₂₃. Consequently, the use of PEO₁₁₄-b-PBCL₁₅ is preferred for delivering CBD when it is desired to minimize the amount of drug formulation to be delivered to a subject.

In Vitro Release Study

An in vitro release study was performed to determine the in vitro release characteristic of free CBD and the above micelle encapsulated CBD formulations (Micellar CBD (DP 23) and d Micellar CBD (DP 15)).

Each of three formulations shown in Table 4 having equal concentrations of CBD were placed in separate Spectra/Por dialysis bags (molecular weight cut-off=12,000-14,000 g mol-1). The dialysis bags were sealed with clips and placed in a beaker with 300 mL 4% bovine serum albumin (BSA), then in a 37° C. shaker. At time 0 and 24 hours, the volume within each bag was measured, and a sample of each solution was taken from inside the dialysis bag and quantified by HPLC. The protocol that was used is described in Aliabadi et al, Journal of Controlled Release (2005) 104 (2), 301-311, which protocol is incorporated herein by reference.

The in vitro release data is contained also in Table 4.

TABLE 4

| Formulation | % CBD released after 24 hours |
|---|---|
| Free-CBD dissolved in acetonitrile | 70.65 |
| Micellar CBD (DP 23) | 2.2 |
| Micellar CBD (DP 15) | 8 |

The results show both micellar formulations to be able to control the release of encapsulated CBD, in the presence of physiological concentrations of serum albumin. This implies that the micellar CBD formulations can be expected to be stable in blood such that the CBD can be carried by the polymeric micelles to fibrotic tissues in the heart. The rapid release of free CBD confirmed the existence of sink conditions in the release experiment. This proves that the conditions of the release experiment are not affecting the observed release of drug from nanoparticles and that the nanoparticles are truly slowing down the release of the encapsulated compound.

Pharmacokinetic Animal Study

An animal study, approved by the Health Sciences Animal Policy and Welfare Committee of the University of Alberta, was conducted to determine the pharmacokinetic profile of micellar CBD (DP 23) and micellar CBD (DP 15) in healthy rats.

Cannulated adult male Sprague-Dawley rats were used for the pharmacokinetic experiments. The animals were housed in a 12-hour light/dark cycle and always had free access to water, although food was withheld for 12 hours prior to drug dosing and 6 hours afterwards.

Rats were divided into 2 groups (n=4/group). Groups 1 and 2 received CBD either encapsulated in PEO-PBCL₂₃ (referred to herein as Micellar CBD (DP 23)) or in free form (CBD dissolved in polyethylene glycol 400 (also referred to as PEG 400)). The administration was by way of subcutaneous injection in the right flank. A single dose of 10 mg/kg was administered to the rats of all groups.

Serial blood samples (200 μL) were collected using jugular vein catheters prior to CBD administration and at 0.25, 0.5, 0.75, 1, 2, 4, 6, 12 and 24, 48 and 72 hours post CBD administration. Plasma was separated (by using a centrifuge) from the samples and kept at −20° C. until it was analyzed for drug content.

Analysis of Plasma Samples for CBD Content

100 μL of plasma samples were analyzed for CBD content as follows. The samples were spiked with internal standard (CBD d3 1 μg/mL) and 400 μL of cold acetonitrile was added to each sample. The mixture was vortexed for 1 minute and then 400 μL of water was added and the resulting mixture was further vortexed for 1 minute. Then 4 mL n-Hexane was added and the resulting mixture further vortexed for 5 minutes. The samples were centrifuged at 1160×g for 15 minutes and the organic phase layers were separated and transferred to clean tubes and evaporated to dryness. The residue was then analyzed for CBD content using a liquid chromatography-mass spectrometry (LC-MS) method involving the use of a Waters Quattro Micro triple quadrupole mass spectrometer. The residue samples were ran in MRM mode using the 315 to 193 transition for CBD, 318 to 196 for Cannabidiol-D₃ solution and 311 to 223 for Cannabinol (CBN). A C18 Agilent Poroshell 120 EC column (2.1×50 mm; 2.7 μm particles) was used. The mobile phase consisted of 0.1% formic acid in HPLC-grade water (Solution A) and 0.1% formic acid in HPLC-grade acetonitrile (Solution B). A gradient elution was programmed to co33mmence with 40% Solution B for post-injection followed by gradual increase in 3 min. of Solution B to 95%. The composition was maintained for 3 min. when was gradually decreased back to 40% of Solution B in 0.1 min. The flow rate was 0.3 mL/min. and 2 μL was injected. The lowest limit of quantification was set at 10 ng/mL.

Pharmacokinetic Parameters

Pharmacokinetic indices were calculated using the non-compartmental method described in J Pharm Pharmaceut Sci (www.cspsCanada.org) 9 (3): 359-364, 2006, which method is incorporated herein by reference.

Data Analysis and Statistics

The pharmacokinetic (PK) data shown is shown in Table 5 (below) and plotted in FIG. 8. In this figure, the data presented is the mean and the error bars depict the standard deviation from the mean. The statistical significance was evaluated using the student's t-test, wherein P-values less than 0.05 were considered significant.

TABLE 5
PK-Parameters for Free and Micellar formulation of CBD.

SC

| | Free-CBD | Micellar CBD |
|---|---|---|
| C_{max (ng/mL)} | 94 ± 86.27 | 17253 ± 11468.6* |
| AUC_{0-72 h} | 687.12 ± 217.39 | 916819.1 ± 619424.6* |
| (ng · h/mL) | | |
| AUC₀₋₆ | 209.45 ± 210.52 | 485.79 ± 96.36 |
| (ng · h/mL) | | |
| tₘₐₓ(h) | 9 ± 10.13 | 25 ± 18 |

*Significantly different from Free drug.

FIG. 8 shows that micellar CBD (DP 23) administered subcutaneously led to a greater amount of CBD entering the bloodstream over 72 hours than subcutaneously administered free CBD solubilized in PEG 300.

The total amount of CBD that entered the bloodstream (AUC₀₋ₜ; 72 h) was calculated using the trapezoidal rule from 0 to the last measured plasma concentration (Clast). The terminal elimination rate constant (β) was estimated using the linear least square regression of the log-linear phase of the concentration-time. Cmax and Tmax were the highest observed concentration and corresponding sampling time point. Table 6 shows that the micellar form of CBD led to greater amounts of CBD entering the bloodstream over six-hour period as compared to free CBD. The difference is even more pronounced over a 72-hour period.

In another pharmacokinetic study (not reported here) employing the same micellar CBD formulation and free CBD formulation used in this experiment, but wherein the formulations were administered intravenously, led to similar results. That is, the micellar CBD formulation led to a greater amount of CBD entering the bloodstream than free CBD, both as a function of time and overall.

One can conclude, based on the above results, that encapsulation of CBD in the micelles using the afore-described block copolymers may lead to more effective delivery of lipophilic active pharmaceutical ingredients, including CBD. With greater amounts of CBD entering the bloodstream using the afore-described technology, an enhancement in therapeutic effect can be expected and that, moreover, when these tests are considered in combination with the tests of Example 4, one can expect that the present block copolymers will be effective to deliver CBD to tissues of the heart, including fibrotic tissues of the heart and to reduce inflammation.

The foregoing description of embodiments is by way of example only and not intended to limit the scope of the invention as described herein and claimed.

1-25. (canceled)

26. A method of treating or preventing heart failure, comprising:
a. identifying a subject suffering from or at risk of developing heart failure; and
b. parenterally administering a micelle comprising a cardioactive agent and an amphiphilic block copolymer to the subject, wherein the amphiphilic block copolymer:
i. comprises a hydrophilic block selected from the group consisting of polyethylene oxide (PEO) (also known as polyethylene glycol (PEG)), polyvinylpyrrolidone (PVP), and derivatives thereof; and a hydrophobic block selected from the group consisting of a poly(ester), a poly(amino acid), a phospholipid, and derivatives thereof; or
ii. is a compound of formula I: 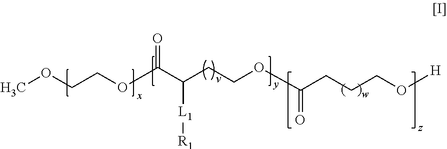
wherein
L₁ is a linker group selected from the group consisting of a single bond, —C(O)—O—, —C(O)—, —O—, —S—, —NH—, —NR²—, and —C(O)NR²;
R¹ is selected from the group consisting of H, OH, C₁₋₂₀ alkyl, C₃₋₂₀ cycloalkyl and aryl, said latter three groups may be optionally substituted and in which one or more of the carbons of the alkyl, cycloalkyl or aryl groups may optionally be replaced with O, S, N, NR² or N(R²)₂ or R₁ is a bioactive agent, typically, cardioactive agent;
R² is H or C₁₋₆ alkyl;
v and w are, independently of each other, an integer independently selected from 1 to 4;
x is an integer from 10 to 300;
y is an integer from 5 to 200;
z is an integer from 0 to 100;
wherein aryl is a mono- or bicyclic aromatic radical containing from 6 to 14 carbon atoms having a single ring or multiple condensed rings; and
wherein the optional substituents are selected from the group consisting of halo, OH, OC₁₋₆ alkyl, C₁₋₆ alkyl, C₂₋₆ alkenyl, C₂₋₆ alkenyloxy, NH₂, NH(C₁₋₆ alkyl), N(C₁₋₆alkyl)(C₁₋₆alkyl), CN, NO₂, C(O)C₁₋₆ alkyl, C(O)OC₁₋₆alkyl, SO₂C₁₋₆ alkyl, SO₂NH₂, SO₂NHC₁₋₆alkyl, phenyl and C₁₋₆ alkylenephenyl;
wherein the micelle passively accumulates in fibrous heart tissue.

27. The method of claim 26, wherein the micelle passively accumulates in cardiac fibroblasts.

28. The method of claim 26, wherein the cardioactive agent is selected from the group consisting of anti-fibrotic agents, anti-inflammatory agents, angiotensin receptor blockers, inotropes, angiotensin II converting enzyme (ACE) inhibitors, cannabidiol, calcium channel blockers, cannabinoids, anti-angiogenic agents, vascular endothelial growth factor (VEGF) antagonists, basic fibroblast growth factor (bFGF) antagonists, bFGF receptor antagonists, transforming growth factor-beta (TGF-β) antagonists, TGF-β receptor antagonists, steroidal anti-inflammatory agents, tumor necrosis factor (TNF) antagonists, VEGF, bFGF, TGF-beta, VEGF receptor antagonists, rapamycin, amiodarone, cyclosporine, cyclosporine A, dobutamine, lipophilic derivatives thereof, and combinations thereof.

29. The method of claim 26, wherein the cardioactive agent is cannabidiol.

30. The method of claim 26, wherein the amphiphilic block copolymer is selected from the group consisting of PEO-polycaprolactone, PEO-poly(valerolactone), PEO-poly(butyrolactone)s, PEO-polylactones, PEO-poly lactides, PEO-polyglycolides, PEO-polylactide-glycolide, PEO-poly(aspartic acid), PEO-poly(glutamic acid), 1,2-distearoyl-sn-glycero-3-phosphoethanolamine-N-[amino(polyethylene glycol) (PEG-DSPE), polyethylene oxide poly(caprolactone (PEO-PCL), poly(ethylene oxide)-block-poly(α-benzyl carboxylate-ε-caprolactone) (PEO-PBCL), poly(ethylene oxide)-block-poly(α-carboxylate-ε-caprolactone) (PEO-PCCL), poly(ethylene oxide)-block-poly(α-cholestryl carboxylate-ε-caprolactone) (PEO-PChCL), and derivatives thereof.

31. The method of claim 26, wherein the amphiphilic block copolymer is selected from the group consisting of poly(ethylene oxide)-block-poly(α-benzyl carboxylate-ε-caprolactone) (PEO-PBCL), poly(ethylene oxide)-block-poly(α-carboxylate-ε-caprolactone) (PEO-PCCL), polyethylene oxide poly(caprolactone (PEO-PCL), and derivatives thereof.

32. The method of claim 26, wherein the micelle has a size selected to localize to cardiac fibroblasts of from about 10, 25, 50, or 70 nm or up to about 500, 250, 200, 175, 150, 125, 100, or 75 nm.

33. The method of claim 26, wherein each of the hydrophobic block and the hydrophilic block has a molecular weight of greater than about 2000, 3000, or 5000 daltons, or up to about 20,000 daltons.

34. The method of claim 26, wherein the heart failure is heart failure with preserved ejection fraction (HFpEF).

35. A micelle for treating or preventing heart failure, the micelle comprising cannabidiol and an amphiphilic block copolymer, wherein the amphiphilic block copolymer:
i. comprises a hydrophilic block selected from the group consisting of polyethylene oxide (PEO) (also known as polyethylene glycol (PEG)), polyvinylpyrrolidone (PVP), and derivatives thereof; and a hydrophobic block selected from the group consisting of a poly(ester), a poly(amino acid), a phospholipid, and derivatives thereof; or
ii. is a compound of formula I: 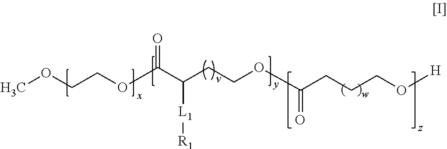
wherein
L₁ is a linker group selected from the group consisting of a single bond, —C(O)—O—, —C(O)—, —O—, —S—, —NH—, —NR²—, and —C(O)NR²;
R₁ is selected from the group consisting of H, OH, C₁₋₂₀ alkyl, C₃₋₂₀ cycloalkyl and aryl, said latter three groups may be optionally substituted and in which one or more of the carbons of the alkyl, cycloalkyl or aryl groups may optionally be replaced with O, S, N, NR² or N(R²)₂ or R₁ is a bioactive agent, typically, cardioactive agent;
R² is H or 01-6 alkyl;
v and w are, independently of each other, an integer independently selected from 1 to 4;
x is an integer from 10 to 300;
y is an integer from 5 to 200;
z is an integer from 0 to 100;
wherein aryl is a mono- or bicyclic aromatic radical containing from 6 to 14 carbon atoms having a single ring or multiple condensed rings; and
wherein the optional substituents are selected from the group consisting of halo, OH, OC₁₋₆ alkyl, C₁₋₆ alkyl, C₂₋₆ alkenyl, C₂₋₆ alkenyloxy, NH₂, NH(C₁₋₆ alkyl), N(C₁₋₆alkyl)(C₁₋₆alkyl), CN, NO₂, C(O)C₁₋₆ alkyl, C(O)OC₁₋₆ alkyl, SO₂C₁₋₆ alkyl, SO₂NH₂, SO₂NHC₁₋₆alkyl, phenyl and C₁₋₆ alkylenephenyl;
wherein the micelle passively accumulates in fibrous heart tissue after parenteral administration thereof to a subject.

36. The micelle of claim 35, wherein the amphiphilic block copolymer is selected from the group consisting of PEO-polycaprolactone, PEO-poly(valerolactone), PEO-poly(butyrolactone)s, PEO-polylactones, PEO-poly lactides, PEO-polyglycolides, PEO-polylactide-glycolide, PEO-poly(aspartic acid), PEO-poly(glutamic acid), 1,2-distearoyl-sn-glycero-3-phosphoethanolamine-N-[amino(polyethylene glycol) (PEG-DSPE), polyethylene oxide poly(caprolactone (PEO-PCL), poly(ethylene oxide)-block-poly(α-benzyl carboxylate-ε-caprolactone) (PEO-PBCL), poly(ethylene oxide)-block-poly(α-carboxylate-ε-caprolactone) (PEO-PCCL), poly(ethylene oxide)-block-poly(α-cholestryl carboxylate-ε-caprolactone) (PEO-PChCL), and derivatives thereof.

37. The micelle of claim 35, wherein the amphiphilic block copolymer is selected from the group consisting of poly(ethylene oxide)-block-poly(α-benzyl carboxylate-ε-caprolactone) (PEO-PBCL), poly(ethylene oxide)-block-poly(α-carboxylate-ε-caprolactone) (PEO-PCCL), polyethylene oxide poly(caprolactone (PEO-PCL), and derivatives thereof.

38. The micelle of claim 35, wherein the micelle has a size selected to localize to cardiac fibroblasts of from about 10, 25, 50, or 70 nm or up to about 500, 250, 200, 175, 150, 125, 100, or 75 nm.

39. The micelle of claim 35, wherein each of the hydrophobic block and the hydrophilic block has a molecular weight of greater than about 2000, 3000, or 5000 daltons, or up to about 20,000 daltons.

40. The micelle of claim 35, wherein the heart failure is heart failure with preserved ejection fraction (HFpEF).

41. The micelle of claim 35, wherein the fibrous heart tissue is cardiac fibroblasts.

42. A composition for treating of preventing heart failure, comprising a micelle according to claim 35 together with a pharmaceutically acceptable carrier.
